SEQUENCE LISTING (5 sequences)

SEQ ID NO: 1 - protein, 749 aa, synthetic construct
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - DNA, 20 bp, synthetic construct
  source 1..20: mol_type = other DNA
accacactgaggacgagatg

SEQ ID NO: 3 - DNA, 20 bp, synthetic construct
  source 1..20: mol_type = other DNA
attgccctcactctcatccc

SEQ ID NO: 4 - DNA, 20 bp, synthetic construct
  source 1..20: mol_type = other DNA
aagccgaggagaaagaagcc

SEQ ID NO: 5 - DNA, 20 bp, synthetic construct
  source 1..20: mol_type = other DNA
tccttcacgaaaccgactcg

(19) United States
(12) Patent Application Publication
(10) Pub. No.: US 2025/0075278 A1
(43) Pub. Date: Mar. 6, 2025

(54) EPIGENTIC REGULATION AND CO-TARGETING APPROACHES OF PSMA IN PROSTRATE CANCER

(71) Applicant: Fred Hutchinson Cancer Center, Seattle, WA (US)

(72) Inventors: Michael Haffner, Seattle, WA (US); Erolcan Sayar, Seattle, WA (US); Michael Schweizer, Seattle, WA (US)

(73) Assignee: Fred Hutchinson Cancer Center, Seattle, WA (US)

(21) Appl. No.: 18/819,098

(22) Filed: Aug. 29, 2024

Related U.S. Application Data
(60) Provisional application No. 63/580,114, filed on Sep. 1, 2023.

(51) Int. Cl. C12Q 1/6886; A61K 45/06; G01N 33/68

(52) U.S. Cl. CPC C12Q 1/6886; A61K 45/06; G01N 33/6893; C12Q 2600/154; G01N 2333/4725; G01N 2333/70596; G01N 2800/52

(57) ABSTRACT

Provided herein are methods and compositions for overcoming resistance to and/or augmenting efficacy of Prostate specific membrane antigen (PSMA)-directed therapies in a subject. In some embodiments, the method comprises administering to the subject in need thereof an effective amount of at least one agent capable of modulating the expression of PSMA. The disclosure also is directed to a method for sensitizing a cancer to a PSMA-targeted therapeutic agent and/or a PSMA-targeted theranostic agent in a subject in need thereof. Also provided are methods and compositions for treating prostate cancer in a subject in need thereof.

### CROSS-REFERENCES TO RELATED APPLICATIONS

This application claims the benefit of U.S. Provisional Patent Application No. 63/580,114, filed Sep. 1, 2023.

### STATEMENT OF GOVERNMENT LICENSE RIGHTS

This invention was made with government support under W81XWH-21-1-0229 awarded by the United States Army Medical Research and Development Command. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P94US_Seq_List_20240827.xml. The XML file is 6,183 bytes; was created on Aug. 27, 2024; and is being submitted electronically via Patent Center with the filing of the specification.

### BACKGROUND

Prostate cancer affects approximately 11% of American men and is the second leading cause of cancer death in the U.S. While the overall 5-year survival rate of men with prostate cancer is nearly 98%, these rates drop significantly to 30% once the cancer has metastasized. The currently available therapeutic interventions for metastatic prostate cancer are highly ineffective at treating the disease, indicating a need for targeted treatment strategies. There is a need in the art for new compositions and methods for treating prostate cancer. The compositions and methods disclosed herein address these and other needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure relates in part to epigenetic regulation and/or modulation of Prostate Specific Membrane Antigen (PSMA) and methods for treating, and/or sensitizing diseases associated with PSMA expression to PSMA-directed therapeutics and/or other therapeutics. In some embodiments, the methods and compositions of the disclosure are used to treat prostate cancer and/or any other cancer associated with low and/or no expression of PSMA.

Accordingly, provided herein is a method for overcoming resistance to and/or augmenting efficacy of Prostate specific membrane antigen (PSMA)-directed therapies in a subject in need thereof. In some embodiments, the method comprises administering to the subject an effective amount of at least one first agent capable of modulating the expression of PSMA. In some embodiments, the subject is human. In an embodiment, the subject has prostate cancer. In some embodiments, the prostate cancer is a PSMA-negative or PSMA-low prostate cancer. In some embodiments, the method is capable of restoring and/or upregulating the expression of PSMA. In some embodiments, the prostate cancer is non-responsive to at least one other therapeutic modality other than PSMA-targeted therapies.

In some embodiments, the at least one first agent capable of modulating the expression of PSMA comprises at least one epigenetic modifier. In some embodiments, the epigenetic modifier targets and/or modulates the epigenetic state of FOLH1 gene to upregulate and/or restore the expression of PSMA. In some embodiments, the epigenetic modifier is selected from a small molecule, a nucleic acid molecule, an expression vector comprising a nucleic acid molecule, and a polypeptide molecule. In an embodiment, the epigenetic modifier is a small molecule. In some embodiments, the small molecule is selected from a histone deacetylase inhibitor (HDACi), NSD2 inhibitor, DNA methyltransferase inhibitor (DNMTi), and combinations thereof. In some embodiments, the small molecule is selected from a combination of (i) histone deacetylase inhibitor (HDACi) and NSD2, (ii) NSD2 and DNA methyltransferase inhibitor (DNMTi), and (iii) HDACi and DNMTi. In an embodiment, the at least one first agent capable of modifying the expression of PSMA is administered prior to or together with administration of or in conjunction with at least one second agent.

In some embodiments, the at least one second agent comprises a therapeutically effective amount of at least one PSMA-directed therapeutic agent. In an embodiment, the at least one PSMA-directed therapeutic agent comprises at least one targeting component coupled to at least one therapeutic component. In some embodiments, the at least one targeting component is selected from a small molecule binder of PSMA, PSMA ligand, PSMA-binding peptide, PSMA-binding aptamer, monoclonal antibodies specific for PSMA, and derivatives thereof. In some embodiments, the at least one therapeutic component or therapeutic agent is a theranostic agent, wherein the theranostic agent is selected from a radioligand/radionuclide or a cytotoxic agent. In some embodiments, the at least one PSMA-directed therapy comprises PSMA-targeted radioligand therapy (PSMA-RLT), wherein the radionuclide is selected from alpha-emitters and beta-emitters.

In another aspect, the present disclosure pertains to a method for treating prostate cancer. In some embodiments, the method comprises: (i) obtaining at least one biological sample from a subject suffering from prostate cancer; (ii) determining the epigenetic state of FOLH1 gene for the presence of at least one epigenetic modification at the FOLH1 gene locus in the at least one biological sample; (iii) determining the expression of at least one of CEACAM5, MUC1, and MSLN in the at least one biological sample; and (iii) administering to the subject at least one agent. In some embodiments, the at least one epigenetic modification is selected from differential methylation of FOLH1 locus, gain of CpG methylation, and loss of histone 3 lysine 27 (H3K27) acetylation, and wherein the at least one epigenetic modification is associated with a low or no expression of PSMA. In some embodiments, the biological sample is determined to overexpress MUC1. In a related embodiment, the at least one agent comprises a therapeutically effective amount of at least one MUC1-targeting therapeutic agent.

In some embodiments, the at least one agent comprises an epigenetic modifier, wherein the epigenetic modifier targets and/or modulates the epigenetic state of FOLH1 gene. In an embodiment, the epigenetic modifier upregulates and/or restores the expression of PSMA. In some embodiments, the epigenetic modifier is a small molecule, and wherein the small molecule is selected from histone deacetylase inhibitor (HDACi), DNA methyltransferase inhibitor (DNMTi), NSD2, and combinations thereof.

In a related embodiment, the method further comprises administering to the subject a therapeutically effective amount of at least one PSMA-targeted therapeutic agent. In an embodiment, the at least one PSMA-targeted therapeutic agent comprises at least one targeting component coupled to at least one therapeutic component. In some embodiments, the at least one targeting component is selected from small molecule binders of PSMA, PSMA ligand, PSMA-binding peptide, PSMA-binding aptamer, and monoclonal antibodies specific for PSMA, and derivatives thereof. In some embodiments, the at least one therapeutic component is selected from a radioligand/radionuclide or a cytotoxic agent. In an embodiment, the at least one agent comprising an epigenetic modifier and the at least one PSMA-targeted therapeutic agent are administered sequentially or simultaneously.

In some embodiments the biological sample is a cell-free sample. In some embodiments, the cell-free sample comprises blood, serum, or plasma. In an embodiment, the prostate cancer is a metastatic castration resistant prostate cancer.

In yet another aspect, provided herein is a method for treating prostate cancer. In some embodiments, the method comprises: (a) selecting and sensitizing a subject in need thereof to a PSMA-directed therapeutic agent; and (b) administering a therapeutically effective amount of at least one PSMA-directed therapeutic agent. In some embodiments, the step of selecting and sensitizing the subject comprises: (i) obtaining a first biological sample from the subject; (ii) determining at least one epigenetic modification of the FOLH1 locus in the first biological sample to obtain a first assessment of PSMA expression; (iii) administering to the subject an effective amount of at least one epigenetic modifier; (iv) obtaining a second biological sample; and (v) determining a change in the at least one epigenetic modification of the FOLH1 locus in the second biological sample to obtain a second assessment of PSMA expression. In some embodiments, the first assessment of PSMA expression comprises a low or no expression of PSMA. In some embodiments, the second assessment of PSMA expression comprises a restoration and/or upregulation of PSMA expression. In some embodiments, the first and the second biological sample is a cell-free sample. In an embodiment, the first and second biological sample is selected from blood, serum, or plasma. In some embodiments, the first and second biological sample comprises blood.

In some embodiments, the at least one epigenetic modification of the FOLH1 locus determined in the first biological sample is selected from differential methylation of FOLH1 locus, gain of CpG methylation, and loss of histone 3 lysine 27 (H3K27) acetylation. In some embodiments, the at least one epigenetic modifier is selected from a small molecule, a nucleic acid molecule, an expression vector comprising a nucleic acid molecule, and a polypeptide molecule. In some embodiments, the at least one epigenetic modifier is a small molecule. In some embodiments, the small molecule is selected from a histone deacetylase inhibitor (HDACi), DNA methyltransferase inhibitor (DNMTi), NSD2, and combinations thereof.

In some embodiments, the at least one PSMA-targeted therapeutic agent comprises at least one targeting component coupled to at least one therapeutic component. In some embodiments, the at least one targeting component is selected from a small molecule binder of PSMA, a PSMA ligand, a PSMA-binding peptide, a PSMA-binding aptamer, and a monoclonal antibody specific for PSMA, and derivatives thereof. In some embodiments, the at least one therapeutic component comprises a radioligand/radionuclide. In an embodiment, the radionuclide is selected from alpha-emitters and/or beta-emitters. In some embodiments, the at least one therapeutic component comprises a cytotoxic agent. In some embodiments, the prostate cancer is a metastatic castration resistant prostate cancer.

In certain embodiments, one or more additional therapeutic modalities may be provided to a subject. In some embodiments, the one or more additional therapeutic modality themselves comprises at least one other therapeutic agent. The one or more other therapeutic modality may be suitable for any kind of disease, but in particular embodiments the disease is prostate cancer. In certain embodiments, the medical condition is metastatic Castration-Resistant Prostate Cancer (mCRPC). In the context of the present disclosure, the one or more additional medical treatment/modalities may comprise administering to the subject at least one of a chemotherapeutic agent, a radiotherapeutic agent, an immunotherapeutic, gene therapy, hormonal therapy, or surgical intervention, for example. The administration of the one or more additional therapeutic modality may precede, follow, or be concurrent with the administration of the (i) at least one epigenetic modifying agent; and (ii) at least one PSMA-targeted/PSMA-directed therapeutic agent; or (iii) at least one MUC1-targeted therapeutic agent disclosed herein.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings, wherein:

FIGS. 1A-1G show PSMA expression patterns differ between molecular subtypes of lethal metastatic prostate cancer. FIGS. 1A-1C show violin plots with distribution of FOLH1 expression (log 2 FPKM) across molecular subtypes (AR+/NE− [green], AR−/NE+ [yellow], AR+/NE+ [red], and AR−/NE− [blue]), determined by RNA-Seq in LuCaP PDX (n=126) (FIG. 1A), SU2C (n=270) (FIG. 1B), and UW-TAN (n=172) tumors (FIG. 1C). FIG. 1D shows representative micrographs of PSMA IHC in different molecular subtypes. Arrows indicate PSMA-positive endothelial cells in cases with absence of tumor cell-specific PSMA expression. FIG. 1E shows a violin plot of PSMA-expression H-scores from the UW-TAN cohort (n=636). FIGS. 1F-1G show correlation plots with a significant positive association (R2 based on Pearson correlation) between PSMA protein expression (by IHC) and FOLH1 mRNA expression (by RNA-seq) in LuCaP (n=25) (FIG. 1F) and UW-TAN samples (n=50) (FIG. 1G). Dot colors indicate molecular phenotypes, as above. Scale bars: 50 μm. *P<0.05; **P<0.001; ***P<0.0001, based on Wilcoxon rank tests.

FIGS. 2A-2K show inter- and intra-patient PSMA expression heterogeneity. FIG. 2A shows dot and box plots for the distribution of PSMA protein expression H-scores in 52 cases from the UW-TAN rapid autopsy cohort (total sample n=636). Each dot represents a tumor sample; the color codes indicate the molecular subtype (AR+/NE− [green], AR−/NE+ [yellow], AR+/NE+ [red] and AR−/NE− [blue]). Gray shadings show interquartile ranges.

FIG. 2B shows a summary of frequencies of cases with uniformly low/negative PSMA expression (all sites H-score≤20), heterogeneous PSMA expression (both H-scores≤20 and H-score >20) and uniformly high PSMA expression (all sites with H-scores ≥20). FIG. 2C shows representative micrographs of PSMA expression patterns in 3 anatomically distinct metastatic sites (all AR+/NE−) from 1 case (case 15-096). FIG. 2D shows PSMA expression in a case with divergent subtypes (case 15-010). FIGS. 2E-2F show the distribution of PSMA expression H-scores across different organ sites in all tumors (FIG. 2E) and AR+/NE− tumors (FIG. 2F). FIG. 2G shows PSMA expression heterogeneity within a metastatic lesion (intra-tumoral heterogeneity). Left panel shows low power (1×) view of a full-face tumor section. Areas with divergent PSMA expression are indicated (FIGS. 2F1-2F3). FIG. 2H shows a high-power view (20×) of 3 tumor foci (FIGS. 2F1-2F3 and from FIG. 2G) showing high level intra-tumoral expression heterogeneity. FIG. 2I shows the mean (95% CI) hypergeometric PSMA expression heterogeneity indices across different metastatic sites in a given patient (intertumoral heterogeneity) and within a metastatic site (intra-tumoral heterogeneity) for the entire cohort (gray) and AR+/NE− tumors (green). FIGS. 2J-2K show the percentage of cases with no (0), 1, 2, 3, 4 or ≥5 metastatic sites with a PSMA H-score≤20 (PSMA-negative metastatic sites). FIG. 2J shows the numbers for the entire cohort (gray bars). FIG. 2K shows the numbers for the AR+/NE− tumors only (green bars). Scale bars: 50 μm.

FIGS. 3A-3F show that PSMA-low/negative tumors have targetable alterations. FIG. 3A shows the heatmap of the top 20 differentially expressed genes with annotated drug target properties from the druggable genome database (rank ordered based on fold expression difference) between PSMA-high and PSMA-low/negative AR+/NE− tumors in UW-TAN, SU2C, and LuCaP PDX cohorts. FIG. 3B shows the top 20 differentially expressed genes encoding for cell surface proteins between PSMA-high and PSMA-low/negative tumors in UW-TAN, SU2C, and LuCaP PDX. Heatmaps are sorted by rank order based on mean fold change differences. FIG. 3C shows representative micrographs of CEACAM5 and MUC1 IHC in PSMA-negative/low and PSMA-high tumors. FIG. 3D shows correlation plots for PSMA, MUC1, and CEACAM5 protein expression. FIG. 3E shows dual fluorescence images of distinct cell population labeling for MUC1 (red) and PSMA (green). FIG. 3F shows heatmaps of PSMA, CEACAM5, MUC1, mesothelin, and CDK6 expression based on IHC H-scores across 289 metastatic sites in 52 patients. Expression scores for each protein target are color coded from light gray to dark gray. White boxes indicate missing data. Each box represents a metastatic site; black boxes outline each case. Scale bars: 50 μm.

FIGS. 4A-4F show epigenetic changes enforce silencing of FOLH1/PSMA. FIG. 4A shows the whole-genome bisulfite sequencing (WGBS) tracks from PSMA-high (LuCaP 77) and PSMA-negative (LuCaP 78 and 93) tumors reveal a differentially methylated region (DMR) encompassing the first 14 kb of the FOLH1 gene and inverse enrichment for H3K27ac. FIG. 4B show the micrographs of PSMA IHC of LuCaP 77, LuCaP 78, and LuCaP 93 demonstrate the difference in PSMA expression. FIG. 4C show representative WGBS tracks of mCRPC tumors from the SU2C-WCDT cohort show gain of methylation in the DMR in PSMA-low/negative tumors. FIGS. 4D-4F show scatter plots of the correlation between FOLH1 expression (based on RNA-Seq) and DMR methylation derived from WGBS (SU2C-WCDT, n=98) (FIG. 4D) and targeted COMPARE-MS analyses UW-TAN (n=18) (FIG. 4E) and LuCaP (n=29) (FIG. 4F) cohorts. Curves were fit by linear regression, and R2 and P values were derived by Pearson correlation.

FIGS. 5A-5I show pharmacologic epigenetic modifiers reverse PSMA silencing. Density plots show PSMA cell surface expression in DU145, LuCaP 35CR cell line (CL), and LAPC4 cells treated for 6 days with vehicle control (DMSO) or 500 nM decitabine (DAC) or 6 days (FIGS. 5A-5B) with panobinostat (PANO, 10 nM) (FIGS. 5E-5F), CUDC-907 (50 nM) (FIGS. 5C-5D) or vorinostat (VOR, 1 μM) (FIG. 5G). Density plot shows PSMA re-expression after treatment with 10 μM of the NSD2 protac MS159 (FIG. 5H). Representative micrographs of cytospin preparations of LuCaP 35CR CL treated with DMSO or panobinostat (10 nM) stained for PSMA and DAPI (FIG. 5I).

FIGS. 6A-6B show enhanced PSMA expression in vivo in mice treated with the HDAC inhibitor CUDC-907. Micrographs of LuCaP 35CR PDX tumors stained for PSMA grown in mice treated with solvent control (30% Captisol) or CUDC-907 at a dose of 75 mg/kg/day for 21 days (FIG. 6A). Percent of PSMA-positive cells in control and CUDC-907 treated tumors (n=4 per group) (FIG. 6B). P-value are based on t-tests. Scale bars indicate 50 μm.

FIGS. 7A-7C show augmented PSMA expression by combined HDAC and DNMT inhibition. Density plots show PSMA cell surface expression in DU145 with vehicle control (DMSO), decitabine (FIG. 7A), Panobinostat (FIG. 7B), or decitabine and Panobinostat (FIG. 7C). Note the right shift of the modal distribution as well as the longer right sided tail in the combination treatment.

### DETAILED DESCRIPTION

While illustrative embodiments have been illustrated and described, it will be appreciated that various changes can be made therein without departing from the spirit and scope of the invention.

“Biological sample” refers to a collection of similar fluids isolated from a subject, as well as fluids present within a subject. Exemplary biological samples are of biological fluids such as blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage, liquid solutions contacted with a subject or biological source, for example, cell and organ culture medium including cell or organ conditioned medium, lavage fluids and the like, tissue biopsies, fine needle aspirations or surgically resected tumor tissue.

A “cancer cell” or a “tumor cell” refers to a cancerous, or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid or uptake of exogenous nucleic acid, it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo (Freshney, Culture of Animal Cells: A Manual of Basic Technique (3rd ed. 1994)).

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is greater.

“PSMA” refers to Prostate Specific Membrane Antigen, a potential carcinoma marker that has been demonstrated to serve as a target for imaging and cytotoxic treatment modalities for cancer.

Embodiments disclosed herein relate to PSMA-targeted or PSMA-directed therapeutic agents. Additional embodiments, relate to the use of compositions or agents including a plurality of PSMA-targeted compounds/agents for the detection and/or treatment of cancer in a subject.

As used herein, the term “small molecule” can refer to lipids, carbohydrates, polynucleotides, polypeptides, or any other organic or inorganic molecules.

“Treat” or “treatment” refer to the treatment of a patient or a subject afflicted with a pathological condition/disease and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included. As used herein, the terms “treating” or “treatment” of a disease can refer to executing a treatment protocol to eradicate at least one diseased (e.g., cancer cell) cell. Thus, “treating” or “treatment” does not require complete eradication of diseased cells.

As used herein, therapeutic agents can include any agent (e.g., molecule, drug, pharmaceutical composition, etc.) capable of preventing, inhibiting, or arresting the symptoms and/or progression of a pathological condition/disease.

As used herein, “an effective amount” is that amount of an agent or a composition that alone, or together with further doses, produces the desired effect, change, and/or biological response, e.g., upregulate or restore expression of PSMA in a cell. As will be appreciated by those of ordinary skill in this art, the effective amount of an agent may vary depending on such factors as the desired biological endpoint, the agent to be delivered, the makeup of the pharmaceutical composition, the target tissue, and the like.

As used herein, “a therapeutically effective amount” refers to amount of a therapeutic agent that results in amelioration of symptoms or a prolongation of survival in the subject and relieves, to some extent, one or more symptoms of the disease or returns to normal (either partially or completely) one or more physiological or biochemical parameters associated with or causative of the disease. A therapeutically effective amount may vary depending on factors such as the disease state, age, sex, and weight of the individual, and the ability of a therapeutic or a combination of therapeutics to elicit a desired response, effect, or change in the individual. Exemplary indicators of an effective therapeutic agent or combination of therapeutic agents that include, for example, improved well-being of the subject as a result of the treatment.

“Restoring the expression”, “upregulating the expression” as used herein in context of PSMA expression refers to a cancer or malignant cell that has measurably higher levels of PSMA expression on the surface compared to the same cell before treatment with an effective amount of at least one agent capable of reversing an epigenetic modification of FOLH1 locus associated with low or no expression of PSMA. PSMA expression can be measured or assessed (assessment of PSMA expression) using well known assays, for example ELISA, immunohistochemistry, immunofluorescence, flow cytometry, western blotting, or radioimmunoassay on live or lysed cells. Alternatively, or additionally, levels of PSMA nucleic acid molecules may be measured in the cell for example using fluorescent in situ hybridization, Southern blotting, or PCR techniques. PSMA expression is restored or upregulated when the level of PSMA on the surface of the cell is at least about 1.5-fold higher when compared to the same cell before treatment with or exposure to an agent.

In certain embodiments, the upregulation or restoration of expression is detectable and greater than that expression in the cell in the absence of exposure to the agent. In specific embodiments, the agent(s) upregulates expression of PSMA at least two-fold, three-fold, four-fold, five-fold, six-fold, seven-fold, eight-fold, nine-fold, ten-fold, twenty-fold, twenty five-fold, thirty-fold, thirty five-fold, forty-fold, forty five-fold, fifty-fold, sixty-fold, sixty five-fold, seventy-fold, seventy-five fold, eighty-fold, eighty five-fold, ninety-fold, ninety-five fold, one hundred-fold, two hundred-fold, five hundred-fold, one thousand-fold, or more as compared to the expression of PSMA in the cell in the absence of exposure to the agent.

In one embodiment, the agent that upregulates or restores expression of PSMA may be of any kind. In specific embodiments the agent is one or both of an epigenetic modifier, including, but not limited to, an HDAC inhibitor and/or a DNMT inhibitor, and/or a NSD2 inhibitor.

As used herein, “epigenetic modifier” refers to an agent that modifies an epigenetic status of a gene in a cell, namely, a phenotype or gene expression in the cell that is caused by mechanisms other than changes in the DNA sequence. An epigenetic status of a gene includes, for example, DNA methylation, histone modification(s) and RNA-associated silencing.

The term “cancer” as used herein refers to an abnormal growth of cells which tend to proliferate in an uncontrolled way and, in some cases, to metastasize (spread).

The term “prostate cancer” as used herein refers to histologically or cytologically confirmed adenocarcinoma of the prostate.

The term “metastatic castration-resistant prostate cancer” refers to castration-resistant prostate cancer (CRPC) that has metastasized to other parts of the human body.

As used herein, the term “subject” is intended to include humans and non-human animals. Preferred subjects include a human characterized by typically low or no expression, of PSMA. Preferred subjects may also include a human patient characterized by an aberrant expression of PSMA and/or at least one of CEACAM5, MUC1, and MSLN. In some embodiments, aberrant expression of PSMA comprises low or no expression of PSMA.

As used herein, the phrase “augmenting efficacy” or “enhancing the therapeutic effects” includes any of a number of subjective or objective factors indicating a beneficial response or improvement of the condition being treated as discussed herein. For example, enhancing the therapeutic effects of at least one therapy in a subject includes upregulating or restoring PSMA expression to sensitize resistant prostate cancer to a PSMA-targeted therapy or enhancing the effects of PSMA-targeted therapy in resistant prostate cancer. In some embodiments, the enhancement includes a one-fold, two-fold, three-fold, five-fold, ten-fold, twenty-fold, fifty-fold, hundred-fold, or thousand-fold increase in the therapeutic efficacy of a therapy.

As used herein, the phrase “overcoming resistance” includes altering or modifying a prostate cancer cell that is resistant to a particular therapeutic intervention such that the cell is no longer resistant to the therapeutic intervention, for e.g., PSMA-directed therapy.

The methods of the present disclosure may be used to treat, prevent, ameliorate and/or screen subjects for a disease associated with PSMA expression. The methods of the present disclosure may also be used to modulate expression of PSMA to overcome resistance and/or sensitize the disease to PSMA-targeted therapeutics. In some embodiments, the disease associated with PSMA expression is selected from the group consisting of prostate cancer, including androgen dependent or androgen independent prostate cancer, and metastases thereof. In another embodiment, the disease includes non-prostate solid tumor. In an embodiment, the non-prostate solid tumors comprise tumors in which PSMA is expressed in the neovasculature of the tumor.

The agents/compounds and/or the compositions described herein can be the agents themselves, pharmaceutically acceptable salts thereof, and pharmaceutically acceptable esters thereof, as well as stereoisomer, enantiomers, racemic mixtures, and the like. The other agent or agents as described herein can be administered as well as a pharmaceutical composition containing the agent(s), wherein the pharmaceutical composition comprises a pharmaceutically acceptable carrier vehicle, or the like.

Reference to an agent/compound or a composition herein applies to the agent or its derivatives and accordingly the disclosure contemplates and includes either of these embodiments (agent; agent or derivative(s)). “Derivatives” or “analogs” of an agent or other chemical moiety include, but are not limited to, compounds that are structurally similar to the agent or moiety or are in the same general chemical class as the agent or moiety. In some embodiments, the derivative or analog of the agent or moiety retains similar chemical and/or physical property (including, for example, functionality) of the agent or moiety

The agents/compounds and/or the compositions of the disclosure, for example the first agent, second agent, and/or the third agent, can be administered by any conventional route, including injection or by gradual infusion over time. The disclosed compounds and additional therapeutic agents, detectable moieties, and/or theranostic agents described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration.

The disclosed agents/compounds or compositions can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a subject or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. When antibodies are used therapeutically, preferred routes of administration include intravenous and by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resorting to undue experimentation.

In specific embodiments, oral, parenteral, or systemic administration are preferred modes of administration.

The compounds or agents disclosed herein can be administered alone as a monotherapy, or in conjunction with or in combination with one or more additional therapeutic agents. The compound/agent can be administered to the subject as part of a pharmaceutical composition comprising the compound/agent and a pharmaceutically acceptable carrier or excipient and, optionally, one or more additional therapeutic agents. The compound and additional therapeutic agent can be components of separate pharmaceutical compositions, which can be mixed together prior to administration or administered separately. The compound/agent can, for example, be administered in a composition containing the additional therapeutic agent, and thereby, administered contemporaneously with the additional therapeutic agent. Alternatively, the agent pr compound can be administered contemporaneously, without mixing (e.g., by delivery of the compound on the intravenous line by which the compound is also administered, or vice versa). In another embodiment, the compound can be administered separately (e.g., not admixed), but within a short time frame (e.g., within 24 hours) of administration of the additional therapeutic agent.

The disclosed compounds and/or an additional therapeutic agents described herein can be administered to the subject in conjunction with an acceptable pharmaceutical carrier or diluent as part of a pharmaceutical composition for therapy. Formulation of the compound to be administered will vary according to the route of administration selected (e.g., solution, emulsion and capsule). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution and Ringer's-lactate. Methods for encapsulating compositions (such as in a coating of hard gelatin or cyclodextran) are known in the art (Baker, et al., “Controlled Release of Biological Active Agents”, John Wiley and Sons, 1986).

Targeting cell surface proteins for therapy and diagnostics has become an important cornerstone in solid tumor oncology. Prostate-specific membrane antigen (PSMA, folate hydrolase I, glutamate carboxypeptidase II) is a 750-residue type II transmembrane glycoprotein highly restricted to prostate secretory epithelial cell membranes. It is highly expressed in prostate cancer cells, benign prostatic tissue, and in nonprostatic solid tumor neovasculature and expressed at lower levels in other tissues, including healthy prostate, kidney, liver, small intestine, and brain. PSMA has a 3-part structure: a 19-amino-acid internal portion, a 24-amino-acid transmembrane portion, and a 707-amino-acid external portion. It forms a noncovalent homodimer that possesses glutamate carboxypeptidase activity based on its ability to process the neuropeptide N-acetylaspartylglutamate and glutamate-conjugated folate derivatives. PSMA is rapidly and efficiently internalized by an endocytic pathway and rapidly recycles back to the membrane. Its large extracellular domain and restricted tissue expression make PSMA a valuable prostate-specific therapeutic target. PSMA is the most extensively validated cell surface target in prostate cancer.

The sequence for the wild type human PSMA (accession NO. Q04609) monomer is shown below (SEQ ID No.: 1).

```
    1 MWNLLHETDS AVATARRPRW LCAGALVLAG
        GFFLLGFLFG WFIKSSNEAT NITPKHNMKA
  61 FLDELKAENI KKFLYNFTQI PHLAGTEQNF
        QLAKQIQSQW KEFGLDSVEL AHYDVLLSYP
121 NKTHPNYISI INEDGNEIFN TSLFEPPPPG
        YENVSDIVPP FSAFSPQGMP EGDLVYVNYA
181 RTEDFFKLER DMKINCSGKI VIARYGKVFR
        GNKVKNAQLA GAKGVILYSD PADYFAPGVK
241 SYPDGWNLPG GGVQRGNILN LNGAGDPLTP
        GYPANEYAYR RGIAEAVGLP SIPVHPIGY
301 DAQKLLEKMG GSAPPDSSWR GSLKVPYNVG
        PGFTGNFSTQ KVKMHIHSTN EVTRIYNVIG
361 TLRGAVEPDR YVILGGHRDS WVFGGIDPQS
        GAAVVHEIVR SFGTLKKEGW RPRRTILFAS
421 WDAEEFGLLG STEWAEENSR LLQERGVAYI
        NADSSIEGNY TLRVDCTPLM YSLVHNLTKE
481 LKSPDEGFEG KSLYESWTKK SPSPEFSGMP
        RISKLGSGND FEVFFQRLGI ASGRARYTKN
541 WETNKFSGYP LYHSVYETYE LVEKFYDPMF
        KYHLTVAQVR GGMVFELANS IVLPFDCRDY
601 AVVLRKYADK IYSISMKHPQ EMKTYSVSFD
        SLFSAVKNFT EIASKFSERL QDFDKSNPIV
661 LRMMNDQLMF LERAFIDPLG LPDRPFYRHV
        IYAPSSHNKY AGESFPGIYD ALFDIESKVD
721 PSKAWGEVKR QIYVAAFTVQ AAAETLSEVA
```

Several PSMA-targeting therapeutics (including radioligand therapies, antibody-drug conjugates, and cell-based immunotherapies) and PSMA-based imaging modalities have been developed. PSMA-positron emission tomography (PET) ligands (68Ga-PSMA-11, 18F-DCFPyL) have rapidly gained a foothold in clinical practice for tumor staging. The recently FDA-approved PSMA-radiopharmaceutical 177Lu-PSMA-617 is one of several potentially novel PSMA-targeting therapies that have demonstrated clinical activity in advanced prostate cancer.

Although PSMA-directed therapeutics have shown highly encouraging clinical activity, a significant percentage of patients do not respond. Not unexpectedly, several recent analyses have shown that the level of PSMA expression is correlated with therapeutic response rates to 177Lu-PSMA-617, suggesting that target expression is a key determinant for the success of PSMA-directed therapies. More broadly, optimal patient selection, assessment of resistance mechanisms, and co-targeting strategies are important to enhance the clinical benefit of PSMA-targeting agents in the future.

Despite the overall enthusiasm for PSMA as a target, relatively little is known about the expression and regulation of PSMA in metastatic castration-resistant prostate cancer (mCRPC). Previous studies have shown that PSMA is expressed in the majority of localized prostate cancers; however, imaging studies of mCRPC have demonstrated that up to 30% of patients have PSMA-negative tumors. A tumor may be defined as PSMA-negative or PSMA-low, if PSMA is not detectable over background noise or is detectable to less than 1.5-fold above the background noise, respectively, utilizing standard methods of detection The small number of prior tissue-based PSMA expression studies in metastatic prostate cancer relied primarily on the evaluation of a single metastatic sample from each patient, which precluded the assessment of the complex expression heterogeneity that may be present across different metastatic sites.

Furthermore, the transcriptional control of PSMA is poorly understood. Although several transcription factors have been implicated in the regulation of PSMA, the molecular mechanisms that contribute to loss of PSMA expression have not been elucidated.

PSMA expression heterogeneity in mCRPC likely poses a critical barrier to the clinical success of PSMA-targeting approaches. This was emphasized by recent preclinical and clinical studies demonstrating that the efficacy of 177Lu-PSMA-617 is tightly correlated with PSMA levels, and high and homogeneous PSMA expression is required for optimal therapeutic response. Therefore, it is important to understand the expression patterns of PSMA to determine and anticipate potential resistance mechanisms of PSMA-directed therapies.

The inventors of the present disclosure comprehensively assessed the inter- and intratumoral heterogeneity of PSMA expression in lethal metastatic prostate cancer and determined the potential mechanisms governing PSMA expression loss to investigate the molecular characteristics of PSMA-negative tumors. Collectively, the data disclosed herein provide novel insights into the expression patterns and transcriptional regulation of PSMA, with important translational implications for PSMA-targeting strategies in mCRPC.

Prior reports have assessed the expression of PSMA in metastatic prostate cancer tissues using IHC and showed absence of PSMA expression in 16%-27% of cases. One limitation of these previous tissue-based PSMA expression studies is that they relied mostly on single-lesion sampling and, therefore, did not provide information on the heterogeneity of PSMA expression between different metastatic sites. To address this issue, the present disclosure determined the expression of PSMA in a cohort of 52 men with lethal metastatic prostate cancer who underwent a rapid autopsy, and metastatic tissues representative of the entire metastatic tumor burden were procured. This unique cohort allowed for the comprehensive assessment of PSMA expression across different metastatic sites in a large number of patients. While PSMA positivity was observed across all metastatic sites in 31% of cases, the vast majority of patients (69%) demonstrated either heterogeneous PSMA expression across different metastatic sites or a complete absence of PSMA labeling. Although AR⁻/NE⁺ tumors consistently lacked PSMA protein expression, it was determined that a substantial number of AR⁺/NE⁻ tumors also showed low levels or absence of PSMA staining. These findings disclosed herein have implications for the clinical implementation of PSMA-targeted therapies and highlight the importance of patient selection. Since lesions with low/negative PSMA expression are likely to contribute to resistance to PSMA-targeting agents, standardized imaging- and tissue-based protocols are needed to screen patients prior to therapy.

In the cohort tested, several tumors that showed no PSMA in tumor cells were positive for neovascular PSMA expression. Although PSMA labeling in tumor-associated vessels has been previously described in other tumor types, the neovasculature of prostate cancer was thought to be less commonly positive for PSMA. The enrichment of PSMA-positive vessels in PSMA-negative tumors raises the possibility of developing vascular targeting strategies. In addition, the neovascular expression of PSMA could account for some of the discrepancies observed between PSMA-PET-based and tissue-based studies. Like previous reports, substantial differences in PSMA expression were observed in different cancer cell clusters within metastatic deposits, demonstrating the high level of intratumoral PSMA expression heterogeneity in mCRPC. Collectively, these findings highlight that in situ assessment of PSMA expression can provide information on the localization and distribution of PSMA expression and emphasize the need to correlate and integrate PSMA-PET imaging with tissue-based analyses in future studies.

Given the relatively high rate of patients with PSMA-negative metastases, the molecular features of these tumors were further characterized. Using 3 independent cohorts (LuCaP PDX, SU2C mCRPC biopsies, and UW-TAN autopsy) and focusing specifically on AR⁺/NE⁻ tumors, it was observed that PSMA-low/negative tumors showed substantially different transcriptomic changes compared with PSMA-expressing tumors. For instance, increased expression in metabolic gene sets, including genes involved in glycolysis, in AR⁺/NE⁻ PSMA-low/negative tumors, was noted. These findings potentially explain observations in prior PET imaging studies, indicating that PSMA-negative tumors can have distinct metabolic activity. Furthermore, an enrichment in inflammatory response and cytokine signaling genes in PSMA-low/negative tumors that was also associated with differences in the composition of the tumor-associated immune microenvironment, with an increase in macrophage infiltration. These observations suggest that PSMA-positive and PSMA-negative tumors broadly exhibit different biological features.

Importantly, several of these biological differences between PSMA-positive and PSMA-negative tumors may be clinically actionable. The analyses disclosed herein revealed additional targets in PSMA-negative tumors—including CEACAM5, MUC1, and MSLN—for which targeting strategies have already been developed. In particular, in silico and in situ studies described herein demonstrate that MUC1 (EMA) could be an attractive target in PSMA-negative tumors, since on the patient, metastasis, and individual tumor cell levels, an inverse correlation between MUC1 and PSMA expression was observed. Of note, MUC1 has been the focus of chimeric antigen receptor T cell (CAR-T) as well as antibody-drug conjugate development efforts for other tumor types. Collectively, the insights presented here pave the way for novel tailored strategies for PSMA-negative tumors using combinatorial approaches that target both PSMA-positive and PSMA-negative cell populations.

In addition to targeting unique alterations in PSMA-negative tumors, understanding the mediators of PSMA silencing would enable the development of strategies to enhance PSMA expression and, thus, augment PSMA-targeting therapies. Therefore, mechanisms underlying transcriptional silencing of PSMA were also explored herein.

In clinical specimens, it was observed that AR-negative tumors tended to have low/absent PSMA levels, suggesting that AR signaling might be required for FOLH1/PSMA expression. This observation contrasts with prior studies demonstrating that, in different model systems, PSMA expression is negatively regulated by AR. The present disclosure shows that, in AR⁺ tumors, AR binding at the FOLH1 locus and AR signaling activity did not significantly differ between PSMA-high and PSMA-low/negative samples. Furthermore, genetic and pharmacologic depletion of AR resulted in a modest increase (not decrease) of PSMA expression. Collectively, these data demonstrate that, although AR can modulate FOLH1/PSMA expression, loss of AR or reduced AR signaling—at least in prostate cancer model systems—does not result in FOLH1 silencing; the data therefore suggest that other mechanisms are likely responsible for the profound changes in PSMA expression observed in CRPC.

To explore alternative modes of PSMA silencing, the epigenetic features of the FOLH1 locus in PSMA-high and PSMA-low/negative models were determined. It was observed that tumors with high PSMA expression exhibited changes in both DNA methylation and histone acetylation. Previous reports have suggested potential epigenetic regulation of PSMA by CpG methylation. Consistent with an earlier study by Zhao et al., it was found that the FOLH1 locus showed gain of CpG methylation in a subset of CRPC cases. However, distinct from the more focal gain of methylation observed in other gene loci, differential methylation of about 14 kb of the FOLH1 locus outside of a CpG island was observed, which was tightly associated with loss of PSMA expression. These findings suggest that epigenetic silencing by CpG methylation contributes to PSMA repression in both PSMA-negative adenocarcinomas and NEPCs. The tight inverse association between CpG hypomethylation and H3K27ac, which is a marker of active enhancers and sites of transcription in PSMA-high tumors, reflects the coordinated interplay between DNA and histone modification.

Notably, none of the cases studied above has undergone PSMA-directed therapies. Therefore, FOLH1 hypermethylation likely represents a potential intrinsic resistance mechanism. It remains to be explored whether epigenetic silencing of PSMA is also a relevant resistance mechanism that arises because of PSMA-directed therapies.

Since epigenetic changes are potentially reversible, different pharmacological epigenetic modifiers were tested for their ability to increase PSMA. Previous studies have demonstrated that HDAC inhibition and demethylating agents can act in concert to induce the expression of silenced genes. Treatment with HDACi (vorinostat, panobinostat, and CUDC-907) resulted in significant upregulation of PSMA expression in vitro and in vivo. While the most consistent re-expression of PSMA was observed with HDACi, DNMT inhibition also showed a modest increase in expression. In addition, differences in the re-expression activity of different HDACi in different cell line models and contexts were observed. These studies demonstrate that epigenetic modifiers can be used to augment PSMA expression.

The present disclosure contemplates a combination of at least one epigenetic modifier, for e.g., HDACi, and at least one PSMA-targeting therapeutic agent to enhance therapeutic efficacy and mitigate primary or secondary resistance due to epigenetic silencing of PSMA. In this context, it is important to note that prior studies have demonstrated a radio-sensitizing effect of HDACi. Therefore, combining HDACi and PSMA-targeting radiopharmaceuticals—in particular, 177Lu-PSMA-617—would augment the radiation-induced therapeutic benefit in addition to inducing increased target expression.

Agents that Upregulate or Restore Expression of a Target Protein

In various embodiments, an agent that upregulates or restores expression of a target protein in a prostate cell is provided. In an embodiment, the target protein is PSMA. In some embodiments, the cell is a prostate cancer cell. In an embodiment, the prostate cancer is a metastatic Castration-resistant Prostate Cancer (mCRPC) cell. In certain embodiments, the upregulation is detectable and greater than that expression in the cell in the absence of exposure to the agent. In specific embodiments, the agent(s) upregulates expression of the target protein at least two-fold, three-fold, four-fold, five-fold, ten-fold, twenty-fold, twenty-five-fold, thirty-fold, thirty-five-fold, forty-fold, forty five-fold, fifty-fold, one hundred-fold, two hundred-fold, five hundred-fold, one thousand-fold, or more.

In one embodiment, the agent that upregulates or restores expression may be of any kind. In specific embodiments the agent is one or both of an epigenetic modifier, including, but not limited to, an HDAC inhibitor and/or a DNMT inhibitor, and a mitogen.

### Epigenetic Modifiers

In some embodiments, one or more epigenetic modifiers are utilized in methods and compositions of the present disclosure to upregulate or restore the expression of at least one protein in a prostate cell. In some embodiments, the cell is a prostate cancer cell. In an embodiment, the prostate cancer is a Castration-resistant Prostate Cancer (CRPC). In some embodiments, the at least one protein comprises PSMA. The one or more epigenetic modifiers may be of any kind so long as they are capable of upregulating and/or restoring expression of the at least one protein. One or more types of epigenetic modifiers may be used in the same method or composition disclosed herein. When more than one epigenetic modifier is employed, they may be delivered to a cell or to a subject at the same time, at different times, in the same formulation, or in different formulations. In some cases, the epigenetic modifier is a histone deacetylase (HDAC) inhibitor. In some cases, the epigenetic modifier is DNA methyltransferase (DNMT) inhibitor. In certain cases, a combination of HDAC inhibitor and DNMT inhibitor is employed. The epigenetic modifier may be an EZH2 antagonist, such as DZnep or 3-deazaneplanocin A, for example.

In some embodiments, the epigenetic modifier(s) comprises one or more histone deacetylase (HDAC) inhibitors. The HDAC inhibitors include, but are not limited to small chain fatty acids, hyroxamic acids, cyclic peptides, or benzamides. Further illustrative examples of HDAC inhibitors include, but are not limited to, trichostatin A, sodium phenylbutyrate, Buphenyl, Ammonaps, Valproic acid, Depakote, romidepsin (ISTODAX®), Vorinostat, Zolinza, panobinostat, belinostat, entinostat, JNJ-26481585 (Johnson & Johnson; Langhorne, Pa.), and/or MGCD-0103 (MethylGene; Montreal, Canada).

In some embodiments, the epigenetic modifier(s) comprises one or more DNMT inhibitors. The DNMT inhibitors include, but are not limited to nucleoside analogs, quinolone, or active site inhibitors. Further illustrative examples of DNMT inhibitors include but are not limited to 5-azacitidine (such as VIDAZA®), decitabine (e.g., DACOGEN®), zebularine, SGI-110 or SGI-1036 (SuperGen; Dublin, Calif.), RG108, caffeic acid purum, chlorogenic acid, epigallocatechin galiate, procainamide hydrochloride, a procainamide derivative, 5-azadeoxycytidine, 5′-aza-2′-deoxycytidine or MG98.

In some embodiments, the one or more epigenetic modifier includes but is not limited to an agent that targets DNA methyltransferases (e.g., DNMT1, DNMT2, DNMT3, DNMT3L), histone acetyltransferases (e.g., GCNS/PCAF, GNAT related, Myst family, CBP/p300, TAF250 family, Src family), histone methyltransferases (e.g., KMT1A, KMT1B, KMT1C, KMT1D, KMT1E, KMT1F, MLL, DOT1, KMT3A, KMT3B, KMT3C, KMT5A, KMT5B, KMT6/EZH2, EZH1, KMT7/SET7&9, KMT8/RIZ1, NSD2), serine/threonine kinases (e.g., MST, AMPK, Haspin, VRK, Aurora A, Aurora B, Aurora C, PLK 1, PLK 2, PLK 3, Chk1, Chk2, ATR, ATM, PKCa/b/d, MSK1/2, JNK1, JNK2, JNK3), MeCP2, MBD1. MBD2, MBD3, bromodomain and extraterminal (BET) proteins (e.g., BRD2, BRD3, BRD4, Bdf, Brg), chromodomain proteins (e.g., HP1-like, polycomb-like, CHD-like), Tudor domain proteins (e.g., SMN), PHD finger proteins (e.g., CBD, ING2, DNMT3L, PHF6), 14-3-3 proteins, MBD2, TET, histone deacteylases (HDAC) Classes I-IV (e.g., HDAC1/2/3/8, HDAC4/5/7/9, HDAC6/10, Sirt1, Sirt2, Sirt3, Sirt4, Sirt5, Sirt6, Sirt7, HDAC11), lysine demethylases (e.g., LSD1/KDM1, JHMD/Jumonji (e.g., JHDM1A/B, JHMD2A/B, JHMD3A-D, JARID1A-D, UTX), protein phosphatases (e.g., PPP2CA, PPP2CB, PPP1C, PP1D, EYA1, EYA2, EYA3), poly (ADP-ribose) polymerase (PARP), hypoxia-inducible factor (HIF), Pim kinases, and Aurora kinases.

### PSMA-Targeted/PSMA-Directed Therapeutic Agents

In some embodiments, the PSMA-targeting/PSMA-directed therapeutic agent comprises at least one “targeting component” coupled to at least one “therapeutic component”. As used herein, the “targeting component” is a component that is able to bind to or otherwise associate with a molecular target or a portion thereof, for example, a membrane component, a cell surface receptor, a cell surface antigen, prostate specific membrane antigen (PSMA, which is also known as folate hydrolase 1, glutamate carboxypeptidase II, and NAALADase), or the like. In an embodiment, the targeting component is able to bind to or otherwise associate with PSMA or a portion thereof. In an embodiment, the targeting component is able to bind to or associate with an extracellular domain of PSMA or a portion thereof. For example, contemplated targeting components that bind to or otherwise associate with PSMA may include a nucleic acid, peptide, polypeptide, protein, glycoprotein, carbohydrate, or lipid. A targeting component may be a naturally occurring or synthetic ligand for PSMA. A targeting component can be an antibody specific for PSMA or portion thereof, which term is intended to include antibody fragments, antibody derivatives, characteristic portions of antibodies, single chain targeting moieties which can be identified, for example, using procedures such as phage display. Targeting components may also be a targeting peptide, targeting peptidomimetic, or a small molecule, whether naturally occurring or artificially created (e.g., via chemical synthesis). In an embodiment the targeting component is selected from the group consisting of an antibody or binding fragment thereof, a protein, a peptide, oligonucleotide, and a small molecule.

As used herein, the “therapeutic component” or the “therapeutic agent” is an agent, or combination of agents, that treats a cell, tissue, or subject having cancer, when contacted with the cell, tissue or subject. Exemplary therapeutic components, include but are not limited to, therapeutic radionuclides, chemotherapeutic agents, hormones, hormone antagonists, receptor antagonists, enzymes or proenzymes activated by another agent, biologics, autocrines or cytokines. In an embodiment the therapeutic component is a theranostic agent. In some embodiments, the therapeutic component may be a cytotoxic agent or a toxin. Other therapeutic components useful in the present disclosure include anti-DNA, anti-RNA, radiolabeled oligonucleotides, such as anti-sense oligodeoxy ribonucleotides, anti-protein and anti-chromatin cytotoxic or antimicrobial agents. Other therapeutic components are known to those skilled in the art, and the use of such other cancer therapeutic components in accordance with the presently disclosed methods is specifically contemplated.

Agents that Target Alternative Molecular Targets in PSMA-Low or PSMA-Negative Prostate Cancer

Embodiments of the present disclosure also contemplate other agents, for example a third agent as disclosed herein that bind to or are specific for an additional molecular target other than PSMA in PSMA-negative tumors. In an embodiment, the additional molecular target is selected from CEACAM5, MUC1, and MSLN. In particular, in silico and in situ studies described herein demonstrate that MUC1 (EMA) is an attractive target in PSMA-negative tumors, since on the patient, metastasis, and individual tumor cell levels, an inverse correlation between MUC1 and PSMA expression was observed. Thus, in certain embodiments, provided herein is at least one MUC1-targeting/MUC1-directed therapeutic agent. In an embodiment, the MUC1-targeting/MUC1-directed therapeutic agent comprises at least one “targeting component” coupled to at least one “therapeutic component” or “therapeutic agent”. In an embodiment, the “targeting component” is a component that is able to bind to or otherwise associate with the molecular target, for example, MUC1 or a portion thereof. A targeting component can be an antibody specific for MUC1 or a portion thereof. The term antibody is intended to include antibody fragments, antibody derivatives, characteristic portions of antibodies, single chain targeting moieties which can be identified, for example, using procedures such as phage display. In an embodiment, the targeting component comprises a chimeric antigen receptor (CAR) comprising a scFv or a humanized variable region that binds to the extracellular domain or a portion thereof of MUC1. In some embodiments, the therapeutic component may be a cytotoxic agent or a toxin. Other therapeutic components or therapeutic agents are known to those skilled in the art, and the use of such other therapeutic agents in accordance with the presently disclosed methods is specifically contemplated.

### Combination Treatments/Other Therapeutic Modalities

In certain embodiments, one or more additional therapeutic modalities may be provided to a subject in addition to the at least one (i) epigenetic modifying agent; (ii) PSMA-targeted/PSMA-directed therapeutic agent; and/or (iii) MUC1-targeted therapeutic agent. In some embodiments, the one or more additional therapeutic modality themselves comprises at least one other therapeutic agent. The one or more other therapeutic modality may be suitable for any kind of disease, but in particular embodiments the disease is prostate cancer. In certain embodiments, the medical condition is metastatic Castration-Resistant Prostate Cancer (mCRPC). In the context of the present disclosure, the one or more additional medical treatment/modalities may comprise administering to the subject at least one of a chemotherapeutic agent, a radiotherapeutic agent, an immunotherapeutic, gene therapy, hormonal therapy, or surgical intervention, for example. The administration of the one or more additional therapeutic modality may precede or follow the administration of the compositions and methods disclosed herein.

Accordingly, in some embodiments, the present disclosure provides a method for overcoming resistance to and/or augmenting efficacy of Prostate specific membrane antigen (PSMA)-directed therapies in a subject. In an embodiment, the subject is human, and the subject has prostate cancer. In some embodiments, the prostate cancer is a PSMA-negative or PSMA-low prostate cancer. In some embodiments, the method comprises administering to the subject an effective amount of at least one first agent capable of modulating the expression of PSMA. In some embodiments, the method restores and/or upregulates the expression of PSMA. In some embodiments, the prostate cancer is non-responsive to at least one other therapeutic modality. In some embodiments, the at least one other therapeutic modality is selected from androgen receptor pathway inhibitors and chemotherapy. In some embodiments, the prostate cancer is metastatic castration resistant prostate cancer (mCRPC).

Also provided herein is a method for treating prostate cancer in a subject in need thereof. In some embodiments, the method comprises administering to the subject at least one first agent effective in modulating the expression of a prostate specific membrane antigen (PSMA); and a second agent comprising a therapeutically effective amount of at least one PSMA-targeted therapeutic agent. In some embodiments, the prostate cancer has a low or no expression of PSMA.

In some embodiments, the at least one first agent capable of modulating the expression of PSMA comprises an epigenetic modifier. In certain embodiments, the epigenetic modifier targets and/or modulates the epigenetic state of FOLH1 gene to upregulate and/or restore the expression of PSMA. In some embodiments, the epigenetic modifier is selected from a small molecule, a nucleic acid molecule, an expression vector comprising a nucleic acid molecule, and a polypeptide molecule. In an embodiment, the epigenetic modifier is a small molecule. In some embodiments, the small molecule is selected from a histone deacetylase inhibitor (HDACi) and DNA methyltransferase inhibitor (DNMTi).

In some embodiments, the at least one first agent capable of modifying the expression of PSMA is administered prior to administration of or in conjunction with a second agent. In an embodiment, the second agent comprises a therapeutically effective amount of at least one PSMA-directed therapeutic agent. In some embodiments, the at least one PSMA-directed therapeutic agent comprises at least one targeting component coupled to at least one therapeutic component. In some embodiments, the at least one targeting component is capable of binding to or associating with PSMA, the extracellular domain of PSMA, or a portion thereof. In an embodiment, the at least one targeting component is selected from a small molecule binder of PSMA, PSMA ligand, PSMA-binding peptide, PSMA-binding aptamer, monoclonal antibodies specific for PSMA, and derivatives thereof. In some embodiments, the at least one therapeutic component or therapeutic agent is a theranostic agent. In an embodiment the theranostic agent is selected from a radioligand/radionuclide or a cytotoxic agent. In some embodiments, the at least one therapeutic component or therapeutic agent is a radionuclide coupled/linked to the targeting component. In an embodiment, the radionuclide is selected from alpha-emitters and beta-emitters. In some embodiments, the at least one PSMA-directed therapy comprises PSMA-targeted radioligand therapy (PSMA-RLT), wherein the PSMA-targeted radioligand therapy is lutetium-177-PSMA-617. In an embodiment, the subject has PSMA-negative or PSMA-low metastatic castration resistant prostate cancer.

In some embodiments, the subject is non-responsive to androgen receptor pathway inhibitors and taxane chemotherapy.

In certain embodiments, the prostate cancer overexpresses at least one of CEACAM5, MUC1, and MSLN. In an embodiment, the prostate cancer overexpresses MUC1, and the method further comprises administering to the subject a third agent. In an embodiment, the third agent comprises a therapeutically effective amount of at least one MUC1-targeting therapeutic agent.

In some embodiments, the first agent, the second agent, and the third agent are administered sequentially, concurrently, or simultaneously. In an embodiment, the first agent is administered prior to the second agent and the third agent. In some embodiments, the second agent and third agent are administered simultaneously. In an embodiment, the first agent is administered prior to the second agent and the third agent. In some embodiments, the second agent and third agent are administered simultaneously.

Further provided herein is a method for sensitizing a cancer to a PSMA-targeted therapeutic agent and/or a PSMA-targeted theranostic agent in a subject in need thereof. In some embodiments, the method comprising: (i) obtaining a first biological sample from the subject; (ii) determining at least one epigenetic modification of the FOLH1 locus in the first biological sample; (iii) administering to the subject an effective amount of at least one first agent comprising an epigenetic modifier; (iv) obtaining a second biological sample from the subject; (v) determining a change in the at least one epigenetic modification of the FOLH1 locus in the second biological sample; and (vi) administering to the subject a therapeutically effective amount of at least one second agent comprising a PSMA-targeted therapeutic.

In some embodiments, the at least one epigenetic modification of the FOLH1 locus in the first biological sample is associated with low or no expression of PSMA. In an embodiment, the change in the at least one epigenetic modification of the FOLH1 locus in the second biological sample comprises a reversal of the at least one epigenetic modification of the FOLH1 locus determined in the first biological sample. In an embodiment, the first and the second biological sample is a cell-free sample.

The present disclosure also provides a method for sensitizing prostate cancer to a PSMA-targeted therapeutic agent in a subject in need thereof. In an embodiment, the method comprises: obtaining a first biological sample from the subject; determining at least one epigenetic modification of the FOLH1 locus in the first biological sample; administering to the subject an effective amount of at least one first agent comprising an epigenetic modifier; obtaining a second biological sample from the subject; determining a change in the at least one epigenetic modification of the FOLH1 locus in the second biological sample; and administering to the subject a therapeutically effective amount of a second agent comprising at least one PSMA-directed therapeutic agent.

In some embodiments, the at least one epigenetic modification of the FOLH1 locus in the first biological sample is associated with low or no expression of PSMA. In some embodiments, the change in the at least one epigenetic modification of the FOLH1 locus in the second biological sample comprises a reversal of the at least one epigenetic modification of the FOLH1 locus determined in the first biological sample. In some embodiments, the first and the second biological sample is a cell-free sample. In an embodiment, the first and the second biological sample comprises blood, serum, or plasma.

In some embodiments, the at least one epigenetic modification of the FOLH1 locus determined in the first biological sample is selected from differential methylation of FOLH1 locus, gain of CpG methylation, and loss of histone 3 lysine 27 (H3K27) acetylation.

In an embodiment, the at least one first agent is selected from a small molecule, a nucleic acid molecule, an expression vector comprising a nucleic acid molecule, and a polypeptide molecule. In certain embodiments, the at least one first agent is a small molecule. In an aspect, the small molecule is selected from a histone deacetylase inhibitor (HDACi), DNA methyltransferase inhibitor (DNMTi), and a histone deacetylase inhibitor (HDACi) and DNA methyltransferase inhibitor (DNMTi).

In some embodiments, the at least one PSMA-targeted therapeutic agent comprises at least one targeting component coupled/linked to at least one therapeutic component. In some embodiments, the at least one targeting component is capable of binding to or associating with PSMA, extracellular domain of PSMA, or a portion thereof. In an embodiment, the at least one targeting component is selected from a small molecule binder of PSMA, a PSMA ligand, a PSMA-binding peptide, a PSMA-binding aptamer, and a monoclonal antibody specific for PSMA, and derivatives thereof. In an embodiment, the at least one therapeutic component comprises a radioligand/radionuclide. In some embodiments, the radionuclide is selected from alpha-emitters and beta-emitters. In some embodiments, the at least one therapeutic component comprises a cytotoxic agent. In some embodiments, the prostate cancer is non-responsive to at least one other therapeutic modality. In some embodiments, the at least one other therapeutic modality is selected from androgen receptor pathway inhibitors and chemotherapy.

In an embodiment, the prostate cancer overexpresses MUC1. In some embodiments, the method further comprises administering to the subject a therapeutically effective amount of a third agent. In some embodiments, the third agent comprises at least one MUC1-targeted therapeutic agent.

In some embodiments, the at least one MUC1-targeted therapeutic agent comprises at least one targeting component coupled/linked to at least one therapeutic component. In some embodiments, the at least one targeting component is capable of binding to or associating with MUC1, extracellular domain of MUC1, or a portion thereof. In an embodiment, the at least one targeting component is selected from a small molecule binder of MUC1, a MUC1 ligand, a MUC1-binding peptide, a MUC1-binding aptamer, and a monoclonal antibody specific for MUC1, Chimeric Antigen Receptor (CAR) T-cell targeting MUC1, and derivatives thereof. In some embodiments, the at least one therapeutic component is a cytotoxic agent.

In yet another aspect, provided herein is a method for treating metastatic castration resistant prostate cancer in a subject in need thereof. In some embodiments, the method comprises: determining at least one epigenetic modification of the FOLH1 locus in a biological sample obtained from the subject; administering to the subject a first agent comprising an effective amount of at least one epigenetic modifier; and administering to the subject a therapeutically effective amount of a second agent comprising at least one PSMA-targeted therapeutic agent. In some embodiments, the metastatic castration resistant prostate cancer has a low or no expression of PSMA. In an embodiment, the metastatic castration resistant prostate cancer expresses at least one other targetable molecule selected from CEACAM5, MUC1, and MSLN.

In some embodiments, the at least one epigenetic modification of the FOLH1 locus is selected from differential methylation of FOLH1 locus, gain of CpG methylation, and loss of histone 3 lysine 27 (H3K27) acetylation. In some embodiments, the first agent comprising at least one epigenetic modifier is effective in reversing the epigenetic modification of the FOLH1 locus to restore and/or upregulate PSMA expression.

In some embodiments, the second agent comprising at least one PSMA-targeted therapeutic agent comprises at least one targeting component coupled/linked to at least one therapeutic component. In some embodiments, the at least one targeting component is capable of binding to or associating with PSMA, extracellular domain of PSMA, or a portion thereof. In some embodiments, the at least one targeting component is selected from small molecule binders of PSMA, PSMA ligand, PSMA-binding peptide, PSMA-binding aptamer, and monoclonal antibodies specific for PSMA, and derivatives thereof. In an embodiment, at least one therapeutic component comprises a radioligand/radionuclide. In some embodiments, the radionuclide is selected from alpha-emitters and beta-emitters. In some embodiments, the at least one therapeutic component comprises a cytotoxic agent.

In some embodiments, the at least one other targetable molecule is MUC1. In an embodiment, the method further comprises administering to the subject a therapeutically effective amount of a third agent comprising at least one MUC1-targeted therapeutic agent.

Provided herein is a method of treating prostate cancer in a subject in need thereof, the method comprising: (i) determining at least one epigenetic modification of the FOLH1 locus in a biological sample obtained from the subject; and (ii) administering to the subject a therapeutically effective amount of an agent comprising a MUC1-targeting therapeutic agent.

In some embodiments, the prostate cancer is a PSMA-negative or a PSMA-low prostate cancer and expresses MUC1. In some embodiments, the biological sample comprises a cell-free sample. In a preferred embodiment, the biological sample comprises blood, serum, or plasma. In some embodiments, the at least one epigenetic modification of the FOLH1 locus determined in the first biological sample is selected from differential methylation of FOLH1 locus, gain of CpG methylation, and loss of histone 3 lysine 27 (H3K27) acetylation. In an embodiment, the method further comprises administering to the subject an effective amount of at least one epigenetic modifier and a therapeutically effective amount of at least one PSMA-targeted therapeutic agent. In some embodiments, the at least one epigenetic modifier is effective in reversing the epigenetic modification of the FOLH1 locus to restore and/or upregulate PSMA expression. In an embodiment, the at least one epigenetic modifier and the at least one PSMA-targeted therapeutic agents are administered to the subject simultaneously or sequentially.

### EXAMPLES

The following examples are included to demonstrate preferred embodiments of the present disclosure. It should be appreciated by those of skill in the art that the techniques disclosed in the examples which follow represent techniques discovered by the inventors to function well in the practice of the disclosed methods and compositions, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Cell lines and in vitro experiments. Human prostate cancer cell lines LNCaP and DU-145 were obtained from the American Type Culture Collection (ATCC). LAPC4, LNCaP95, and LNCaP95 AR-KO cells were gifts from John Isaacs (Johns Hopkins School of Medicine, Baltimore, Maryland, USA). The LuCaP 35CR cell line (35CR CL) was derived from the LuCaP 35CR PDX model and provided by Peter Nelson (Fred Hutchinson Cancer Center). All cells were grown in the recommended media supplemented with 10% FBS (Sigma-Aldrich) and maintained at 37° C. with 5% CO2. Short tandem repeat genotyping was used to authenticate all lines, and cells were confirmed to be mycoplasma free using the MycoAlert Detection Kit (Lonza, LT07-418). Cells were cultured for no more than 10 passages after thawing and before experimental use. ENZA, decitabine, vorinostat, panobinostat, and CUDC-907 were purchased from SelleckChem; MS159 was purchased from MedChem. All drugs were diluted in DMSO. For in vitro experiments, cells were seeded at 300,000 per well in 6-well plates and treated with 2 doses of inhibitor 3 days apart. Cells were collected 6 days after the first dose for flow cytometric analysis or fixed for 2 hours in formalin and spun down on slides for immunofluorescence analysis. To assess PSMA cell surface expression by flow cytometry, cells were dissociated, washed once with FACS buffer (PBS+10% FBS), and stained with PE anti human PSMA antibody (BioLegend, 342504) by resuspending cells in 100 μL of FACS buffer and in 5 μL of PE anti-PSMA antibody and by incubating cells on ice in the dark for 20 minutes. Cells were washed 3 times with FACS buffer before analysis on a Sony SH800 cell sorter (Sony Biotechnology). All downstream analyses were performed using FlowJo (v10). For IHC studies, cells were collected by trypsinization, fixed for 2 hours in 10% buffered formalin, and used for cytospin preparation as described previously.

### Example 2

In vivo experiments. All surgical procedures were performed under isoflurane anesthesia. LuCaP 35CR tumors (1 mm3) were surgically implanted s.c. into castrated male CB17SCID mice (The Jackson Laboratory) (59). When tumors reached a volume between 150 mm3 and 200 mm3, mice were administered CUDC-907 (Curis Inc.), a combined pan-HDAC/PI3K inhibitor dissolved in 30% captisol (Thermo Fisher Scientific). Treated mice received 75 mg/kg/day for 5 days, followed by 2 days off treatment, repeated for a total of 21 days or until the diameter of the tumor reached 2 cm. The same volume of 30% captisol, in the same manner as described above, was used to treat the vehicle group. At the end of the experiment, mice were euthanized, and tumors were harvested and fixed in 10% formalin before IHC analyses.

### Example 3

Human tissue samples. Metastatic cancer samples were collected as part of the Prostate Cancer Donor Program at the UW, and tissue microarrays (TMA) containing 52 patient samples from available tissues specimens from different metastatic sites (median number of sites per patient, 7; range, 1-21) were constructed as described previously. None of these patients received prior PSMA-directed therapies.

### Example 4

IHC staining. For chromogenic PSMA IHC staining, slides were deparaffinized and steamed for 45 minutes in Target Retrieval Solution (Agilent/Dako). The primary PSMA antibody (Agilent, M3620, clone 3E6) was used at 1:50 dilution. Immunocomplexes were detected using PV poly-HRP anti-mouse IgG (Leica Microsystems, PV6114) with DAB as the chromogen. For MUC1, CEACAM5, MSLN, and CDK6 staining, the following antibodies and pretreatment conditions were used: MUC1 (target retrieval solution, Agilent, M0613, clone E29, 1:20), CEACAM5 (target retrieval solution, Agilent, M7072, clone II-7, 1:20), MSLN (target retrieval solution, MilliporeSigma, 439R-1, 1:15), and CDK6 (antigen unmasking solution, Vector Labs, H-3300-250; Abcam, ab124821, 1:25). PV Poly-HRP anti-mouse IgG (Leica Microsystems, PV6114) and anti-rabbit IgG (Leica Microsystems, PV6119) were used as secondary antibodies. Immunocomplexes were detected using the Biotin XX Tyramide SuperBoost kit (Invitrogen, B40931) per manufacturer's protocol with DAB as the chromogen. For AR and NE status assessment, antibodies specific to AR (Cell Signaling Technology, 5153T, 1:100), NKX3.1 (Thermo Fisher Scientific, 5082788, 1:50), synaptophysin (Thermo Fisher Scientific, RM9111S, 1:80), and INSMI (Santa Cruz Biotechnology Inc., A-8, SC271408, 1:100) were used according to protocols described previously. For dual-immunofluorescence labeling of MUC1 and PSMA, a sequential staining protocol was used. MUC1 (Agilent, M0613) and PSMA (Agilent, M3620) were used at 1:20, followed by PV poly-HRP anti-mouse IgG (Leica Microsystems, PV6114). Target retrieval solution (Agilent Technologies, S169984-2) was used for antigen retrieval and antibody stripping. Immunocomplexes were detected using Tyramide Signal Amplification system from Thermo Fisher Scientific. For chromogenic IHC, tissue sections were counterstained with hematoxylin, and the slides were digitized on a Ventana DP 200 Slide Scanner (Roche). Membranous PSMA expression was scored in a blinded manner by 2 pathologists, whereby the optical density level (“0” for no brown color, “1” for faint and fine brown chromogen deposition, and “2” for prominent chromogen deposition) was multiplied by the percentage of cells at each staining level, resulting in a total score range of 0-200. The final score for each sample represents the average of 2 duplicate tissue cores. For immunofluorescence studies, Alexa Fluor 568 Tyramide (Invitrogen, B40956) was used for signal amplification. Slides were counterstained with DAPI (Thermo Fisher Scientific) and mounted with Prolong (Thermo Fisher Scientific), and fluorescence images were captured using a Nikon Eclipse E800 microscope (Nikon).

### Example 5

In silico expression analysis. RNA-Seq data of bulk flash-frozen tissues from the SU2C/Prostate Cancer Foundation, LuCaP PDXs, and UW-TAN mCRPC cohorts were processed as described previously. All subsequent analyses were performed using R. Gene abundance was quantified using GenomicAlignments. Molecular subtype classification (AR/NE status) was performed as described previously. Tumors were assigned to PSMA-low and PSMA-high categories by dividing them into those with PSMA expression below and above the mean across tumors for each data set. The data sets were then reduced to only include AR+/NE− tumors. Differential expression between PSMA-low versus PSMA-high groups was assessed using limma and was filtered for a minimum expression level using the filterByExpr function with default parameters prior to testing and using the Benjamini-Hochberg FDR adjustment. Results were then filtered to genes with FDR≤0.05 and absolute value fold change >2 in at least 2 data sets. These were further refined to cell surface and tier 1 druggable targets with the same thresholds in at least 1 data set. Genome-wide gene expression results were ranked by their limma statistics and used to conduct GSEA to determine patterns of pathway activity utilizing the curated pathways from MSigDBv7.4. Single-sample enrichment scores were calculated using GSVA with default parameters using genome-wide log 2 FPKM values as input, the 10-gene androgen-regulated signature and 31-gene cell cycle proliferation (CCP) scores. Immune decomposition was performed using CIBERSORTx with LM22 cell type signatures, B-mode batch correction, absolute mode, and 1,000 permutations. Tumors were assigned to PAM50 categories using a previously described classification method. Classification was restricted to luminal A, luminal B, and basal, removing Her2 and normal samples from the training set and centroid scores prior to classification. Groups displayed in box plots were compared using 2-sided Wilcoxon rank tests with Benjamini-Hochberg multiple-testing correction.

### Example 6

DNA methylation analyses. For WGBS analyses, DNA was extracted from LuCaP PDX lines (e.g., 77, 78, and 93), subjected to bisulfite conversion, and sequenced on an Illumina HiSeq 2500 instrument (Illumina) to an average coverage of 30×. Raw WGBS reads were first trimmed using Trim Galore (0.6.6) and then aligned to UCSC hg19 reference genome using Bismark (0.23.0). Bismark was further used to deduplicate the alignments and extract methylation call files, which report the percentage of methylated cytosines and the coverage at each position. WGBS data are publicly available on Gene Expression Omnibus (GEO; accession no. GSE205056). A previously validated assay combining methylated-DNA precipitation and methylation-sensitive restriction enzyme digestion (COMPARE-MS) was used for site-specific DNA methylation analyses. In brief, DNA samples were digested with Alul and Hhal (New England Biolabs), and methylated DNA fragments were enrichment using recombinant MBD2-MBD (Clontech) immobilized on magnetic Tylon beads (Clontech). The precipitated DNA containing methylated DNA fragments was eluted and subjected to quantitative PCR (qPCR) using IQ SYBR Green Supermix (Bio-Rad) with primers specific to the second intron of FOLH1 (hg19 chr11:49228686-49228864) forward (F): 5′-ACCACACTGAGGACGAGATG-3′; reverse (R): 5′-ATTGCCCTCACTCTCATCCC-3′. For quantitative assessment of locus-specific methylation levels, Ct values of the samples of interest were normalized to Ct values of the positive control (in vitro fully methylated male genomic DNA), and methylation indices were calculated (range, 0%-100%).

### Example 7

Statistics. Distributions of H-scores across molecular subgroups, defined by AR signaling and NE marker expression, were compared using the Kruskal-Wallis test. Associations between H-scores and FOLH1 mRNA levels in LuCaP PDX and UW-TAN samples were evaluated using linear regression. Heterogeneity of subgroups across metastatic sites in a given patient was quantified using hypergeometric probabilities that a randomly chosen pair of sites have different subgroups. Alternative measures of heterogeneity and Shannon and Simpson indices were obtained using the R package Vegan. Differences in distributions of heterogeneity scores in the UW-TAN for patients that died in 2003-2010 versus 2011-2019 were visualized using kernel density estimation and compared using Kolmogorov-Smirnov tests. Heterogeneity across metastatic sites in a given patient (intertumoral heterogeneity) and within a metastatic site (intratumoral heterogeneity) was summarized across patients using the mean outcome of 1,000 randomly sampled pairs and bias-corrected and accelerated 95% CI limits from the R package Bootstrap. In all analyses, P<0.05 was considered statistically significant.

Study approval. In vivo studies were approved by the FHCC IACUC (IR 51048) and were performed in strict accordance with the guidelines in the Guide for the Care and Use of Laboratory Animals of the National Institutes of Health. Human tissue studies were approved by the IRB of the UW (protocol no. 2341). All rapid autopsy tissues were collected from patients who provided written informed consent under the aegis of the Prostate Cancer Donor Program at the UW.

### Example 8

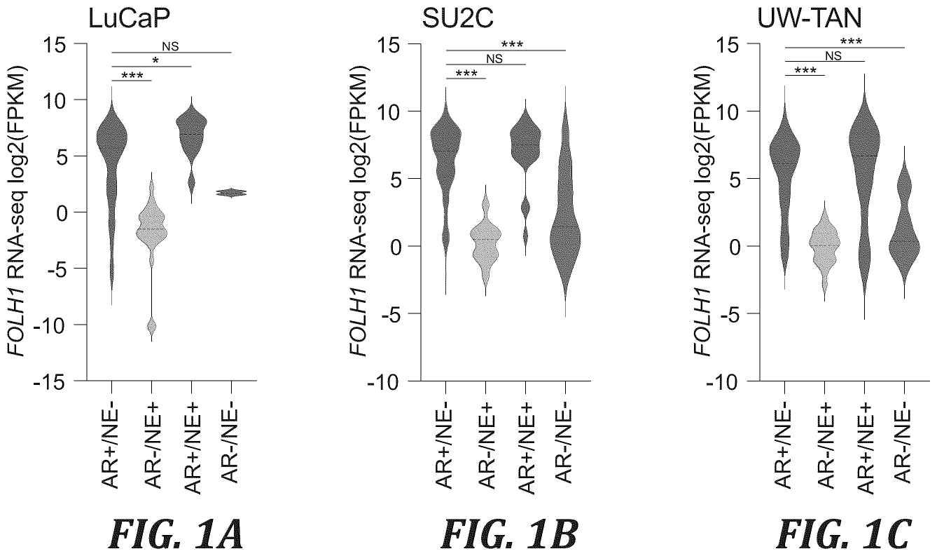

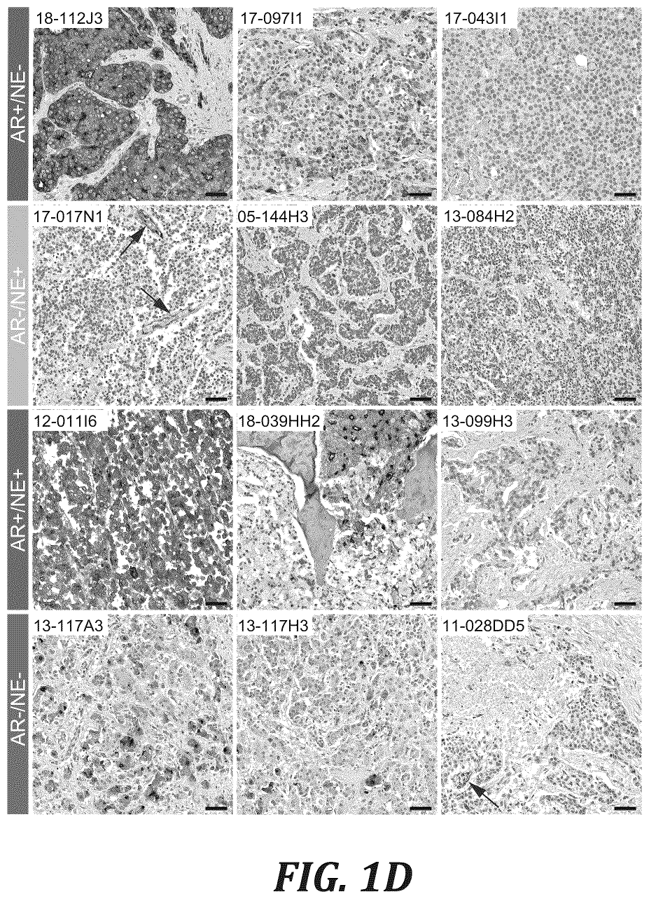

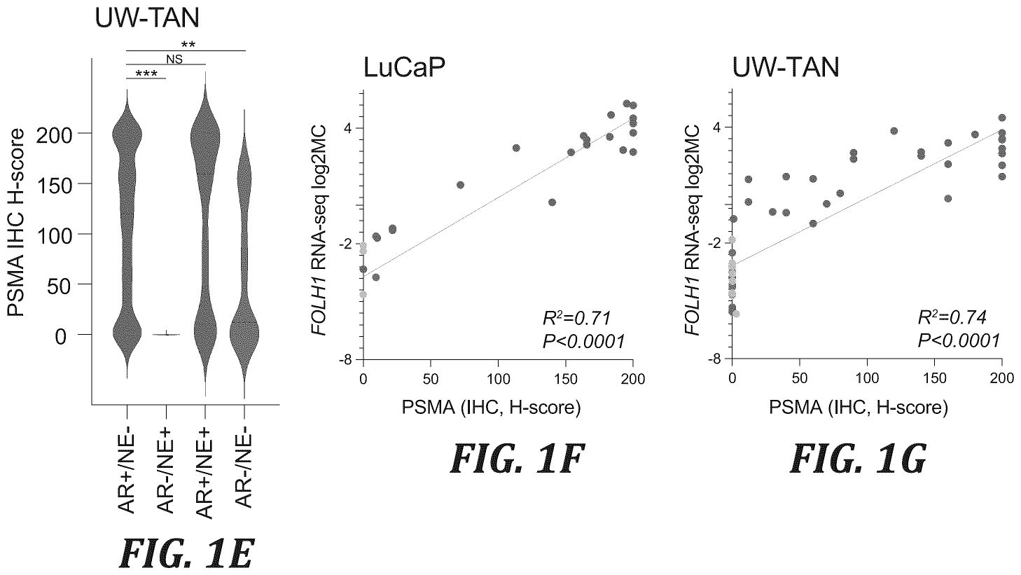

PSMA expression differs across molecular subtypes of prostate cancer. Recent studies have suggested that mCRPC can be divided into 4 molecular subgroups based on androgen receptor (AR) signaling and neuroendocrine (NE) marker expression. To determine the expression of PSMA in these clinically relevant subsets, FOLH1 mRNA levels were assessed in 3 cohorts comprising 126 LuCaP prostate cancer patient-derived xenograft (PDX) samples (FIG. 1A), 270 Stand Up To Cancer (SU2C) international dream team mCRPC biopsies (FIG. 1B), and 172 lethal metastatic prostate cancer samples from the UW rapid autopsy program (UW Tissue Acquisition Necropsy [UW-TAN]) (FIG. 1C). A high level of expression variability was observed in AR+/NE− tumors (mean across cohorts, 48.5 fragments per kilobase of exon per million mapped fragments [FPKM]; range, 0.02-1269). Similar to prior reports, low/negative PSMA expression was found in AR−/NE+ tumors (NE prostate cancer [NEPC]) (mean across cohorts, 0.59 FPKM; range, 0.0008-8.26) (27). AR+/NE+ showed expression levels comparable to AR+/NE− tumors (mean across cohorts, 84.33 FPKM; range, 0.3-698.4), whereas AR−/NE− tumors demonstrated reduced levels (mean across cohorts, 3.40 FPKM; range, 0.3-363.6) (FIGS. 1A-1C) and AR activity signature scores correlated with PSMA expression.

To further determine PSMA protein expression in mCRPC, PSMA IHC was performed on 636 samples from 339 anatomically distinct metastatic sites of 52 cases of the UW-TAN cohort using a previously validated IHC assay (FIG. 1D). Protein expression levels differed substantially between AR+ and AR− tumors (AR+/NE−: mean H-score, 106.9; AR+/NE+: mean H-score, 97.7; AR−/NE+: mean H-score, 0.06; AR−/NE−: mean H-score, 50.4) (FIGS. 1D-1E). Importantly, while there was a general trend to lower PSMA expression in AR-tumors, several AR−/NE− cases were identified with relatively high PSMA H-scores (FIG. 1E). Similar to the mRNA levels, a significant number of tumors showed no (103 of 339; 30%) or low (H-score≤20) (137 of 339; 40%) PSMA expression. PSMA protein expression levels appeared to follow a bimodal distribution, with the largest groups of samples showing either low or high H-scores. In subset analyses of AR+/NE− tumors, it was found that 47 of 204 (23%) were low/negative for PSMA. Notably, focal PSMA staining of the tumor-associated vasculature was observed in tumors lacking PSMA expression in tumor cells (FIG. 1D). To better understand the association between PSMA protein expression (determined by IHC) and FOLH1 mRNA transcript levels, LuCaP PDX and UW-TAN samples were analyzed with matched IHC and RNA-Seq data (FIG. 1F-1G). A strong correlation between protein and mRNA expression was observed, demonstrating that mRNA levels can be used as a proxy for assessing PSMA protein expression.

These data document the diversity of PSMA expression across different molecular subtypes and highlight the high level of overall expression variability in mCRPC.

### Example 9

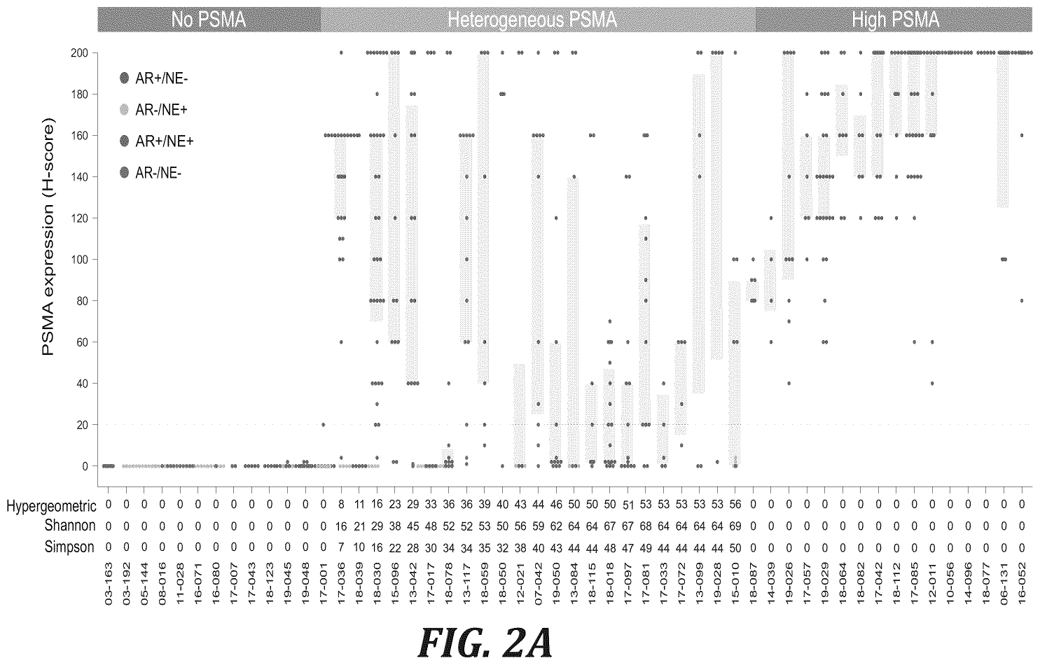

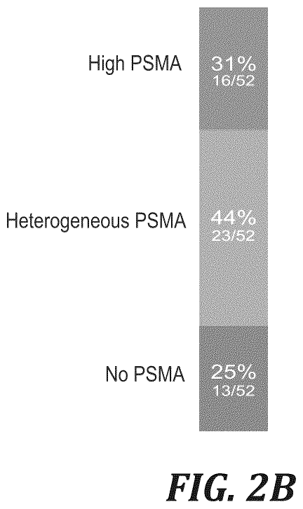

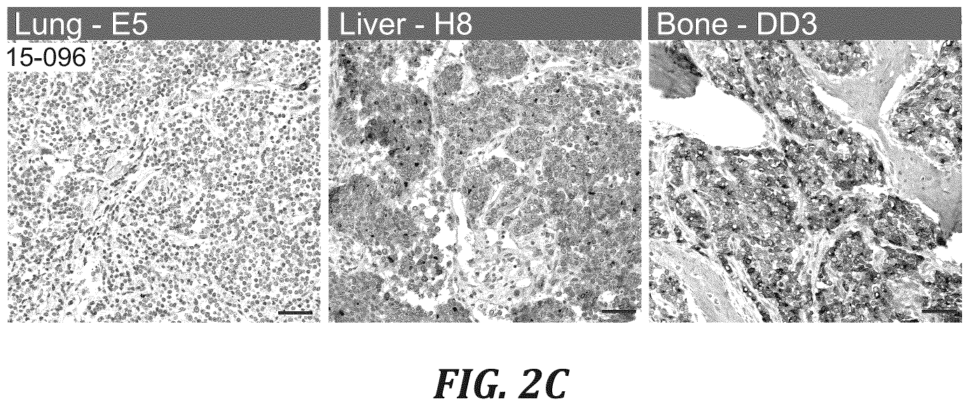

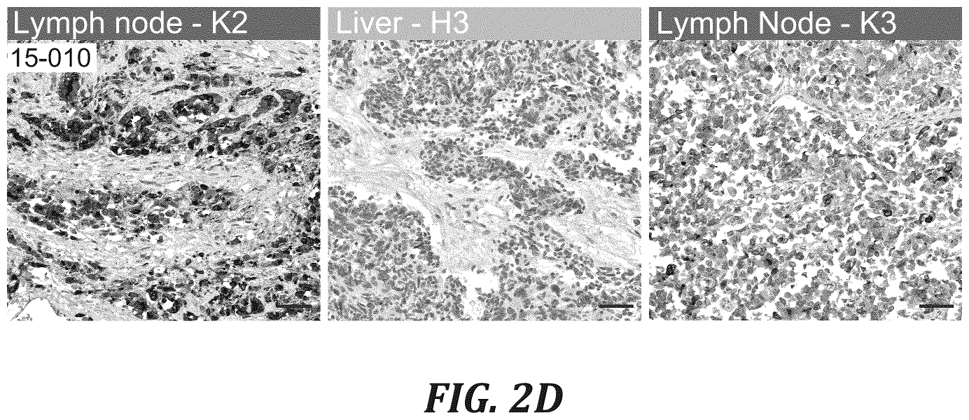

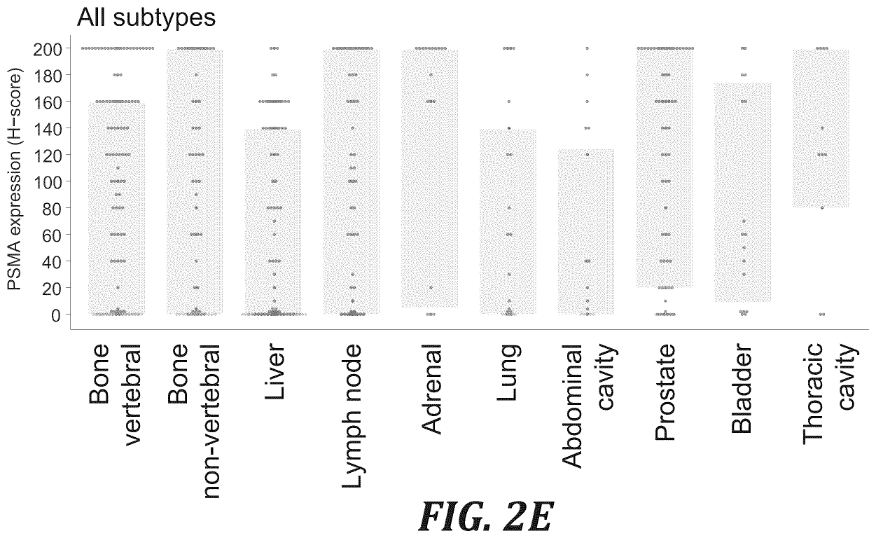

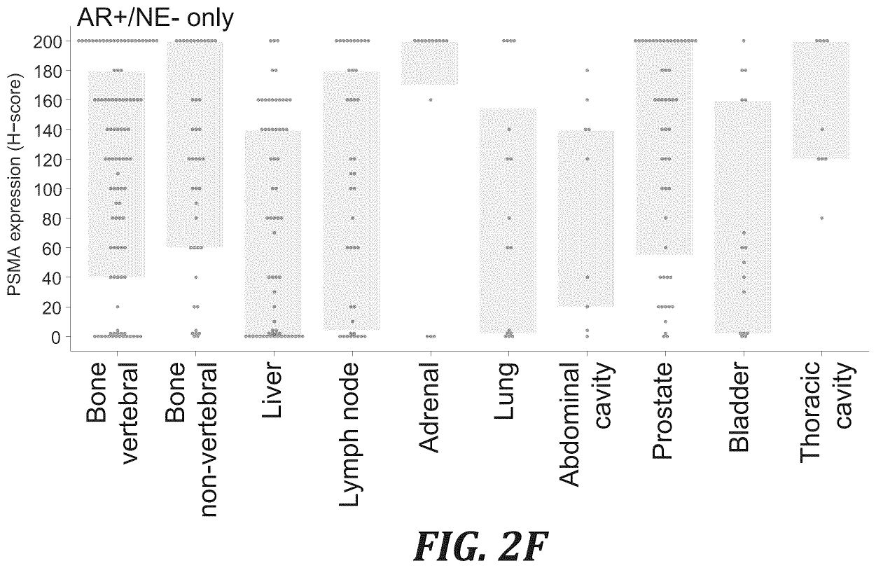

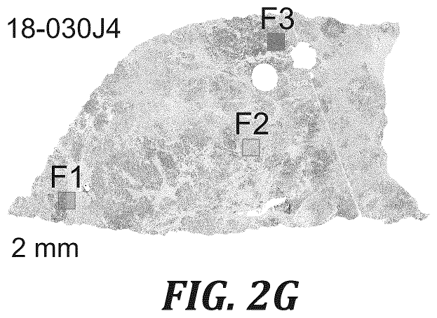

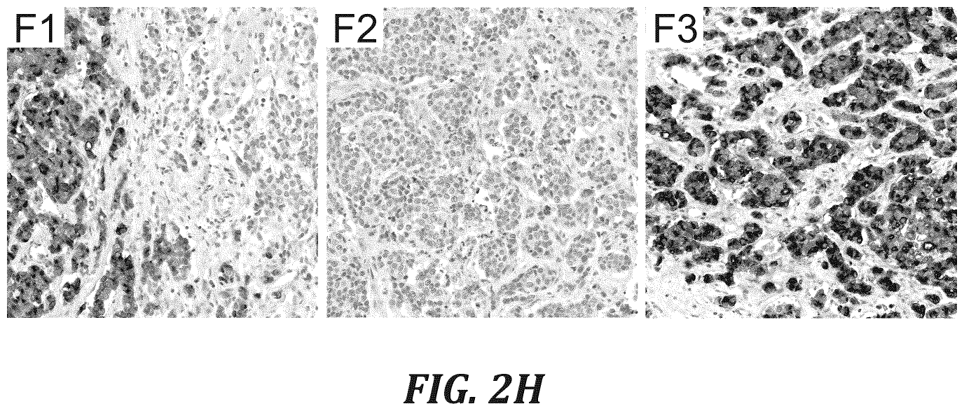

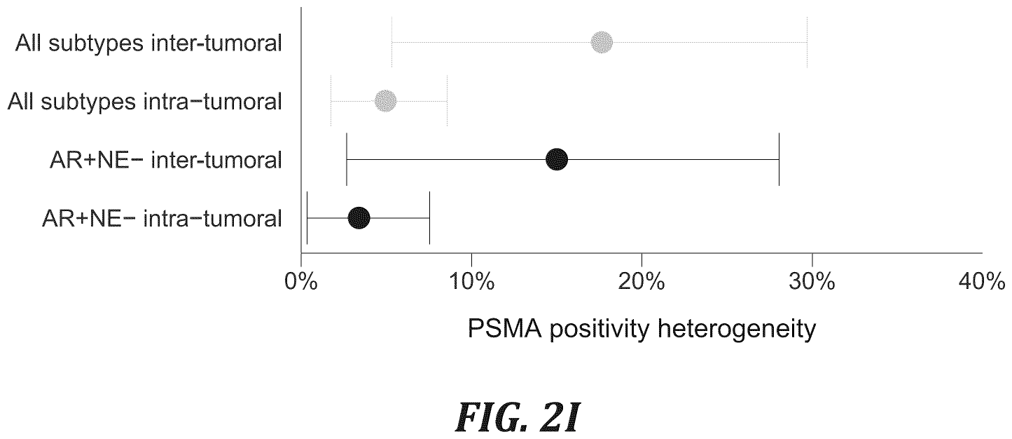

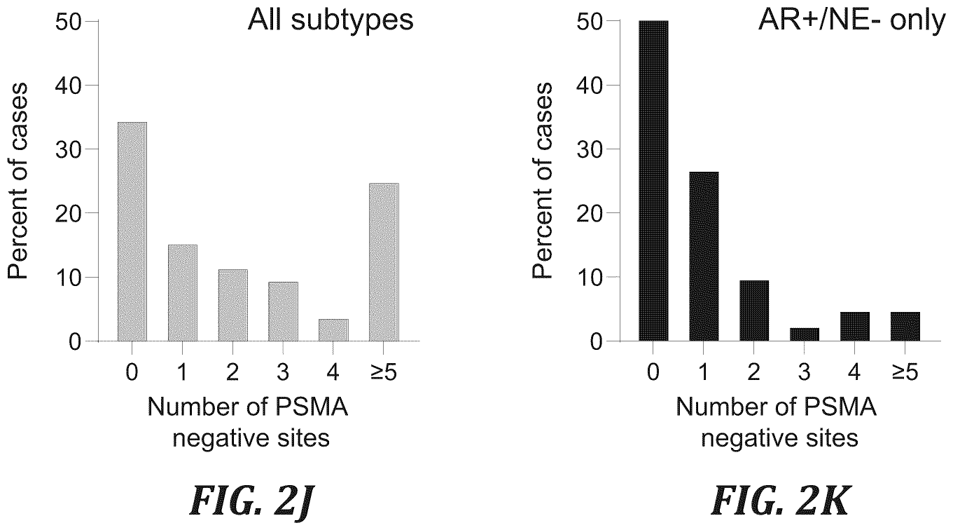

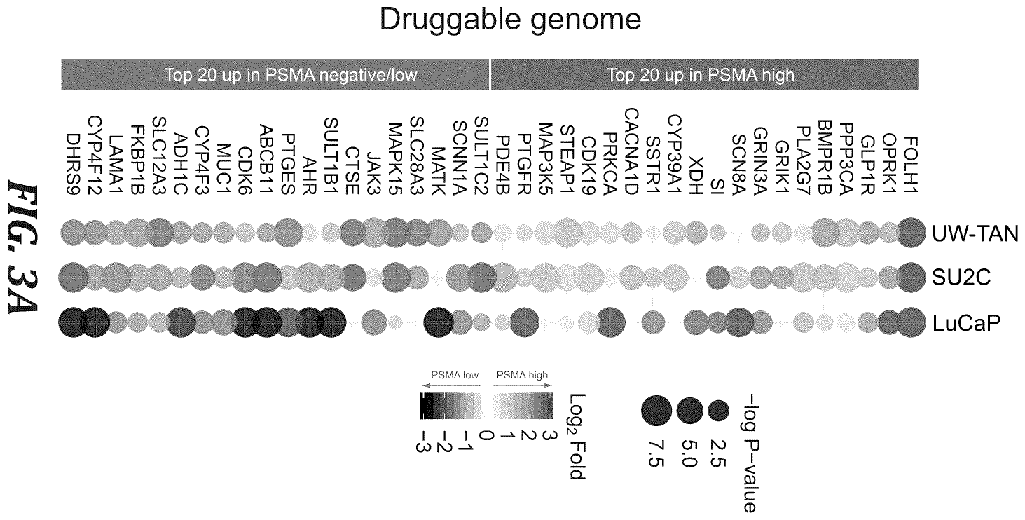

PSMA expression shows a high level of inter- and intra-tumoral heterogeneity. To further study inter- and intra-patient PSMA expression variability, the unique tissue resources and design of the UW-TAN rapid autopsy cohort allowed for multiple metastatic sites from each patient to be sampled. From this, both intertumoral (between different metastatic sites) and intra-tumoral heterogeneity (between different cores from one metastasis) were determined. An H-score of ≤20 was defined as the cutoff for tumors with low/negative PSMA expression. Across the 339 tumors from 52 cases in this cohort, 3 patterns (low/negative, heterogenous, uniformly high) of PSMA expression were observed (FIGS. 2A-2B). Thirteen of 52 (25%) cases showed low/negative PSMA expression across all metastatic sites (FIGS. 2A-2D). Although this group was enriched for AR−/NE+ tumors, it also included several AR+/NE− cases. Patients with heterogeneous PSMA expression (defined by the presence of lesions with low/negative PSMA [H-score≤20] and high PSMA [H-score >20]) composed the largest group (23 of 52; 44%). This group included cases with variable PSMA in metastases of uniform molecular subtype (e.g., case 15-096; FIGS. 2A-2D) as well as cases with divergent molecular subtypes in different anatomic sites (e.g., case 15-010; FIGS. 2A-2D). Trends toward lower PSMA H-scores in liver metastases (mean, 66; range, 0-200) and higher PSMA levels in adrenal (mean, 118; range, 0-200) and prostate tumors (mean, 106; range, 0-200) were observed compared with bone sites (vertebral and nonvertebral combined; mean, 88; range, 0-200; P=0.001 and P=0.02, respectively), when including all molecular subtypes (FIG. 2E). These trends persisted in subset analyses of AR+/NE− tumors (FIG. 2F). These findings suggest a potential interplay between the tumor microenvironment of metastatic sites and PSMA expression.

In addition to intertumoral heterogeneity, substantial intra-tumoral differences in PSMA expression were also noted (FIGS. 2G-2H). In full face tissue sections, spatially separate cell clusters with distinct PSMA expression were identified. This demonstrated that PSMA-high and PSMA-negative cell populations exist within a given metastasis (FIGS. 2G-2H).

To formally quantify the variability in PSMA expression, 3 measures of heterogeneity were examined (hypergeometric probabilities and Shannon and Simpson indices; FIG. 2A and FIG. 2I). To account for PSMA expression heterogeneity driven by molecular subtype-specific expression patterns, additional subset analyses on AR+/NE− tumors were performed. PSMA heterogeneity across different metastatic sites in a patient (intertumoral) and within an individual metastatic site were assessed (intra-tumoral) (FIG. 2I). Similar levels of PSMA expression heterogeneity were observed in AR+/NE− tumors (estimated probability of intertumoral differences, 15% [95% CI, 3%-28%]; estimated probability of intra-tumoral differences, 3% [95% CI, 0%-8%]) compared with the entire unselected cohort (estimated probability of inter-tumoral differences, 18% [95% CI, 5%-30%]; estimated probability of intra-tumoral differences, 5% [95% CI, 2%-9%]) (FIG. 2I). Next, the frequency of PSMA-low/negative metastases were determined. In unselected patients of all molecular subtypes, only 18 of 52 (34.6%) showed PSMA expression (H-scores >20) in all metastatic sites (FIG. 2J). In contrast, 21 of 41 (51.2%) patients with AR+/NE− tumors showed PSMA expression in all metastatic sites, whereas 2 of 41 (4.9%) had 5 or more PSMA-negative sites (FIG. 2K). PSMA expression heterogeneity indices were slightly decreased in patients treated prior to the widespread use of abiraterone and enzalutamide (ENZA); however, this trend was not statistically significant. These findings suggest that treatment with second-generation AR signaling inhibitors does not profoundly alter PSMA expression patterns.

### Example 10

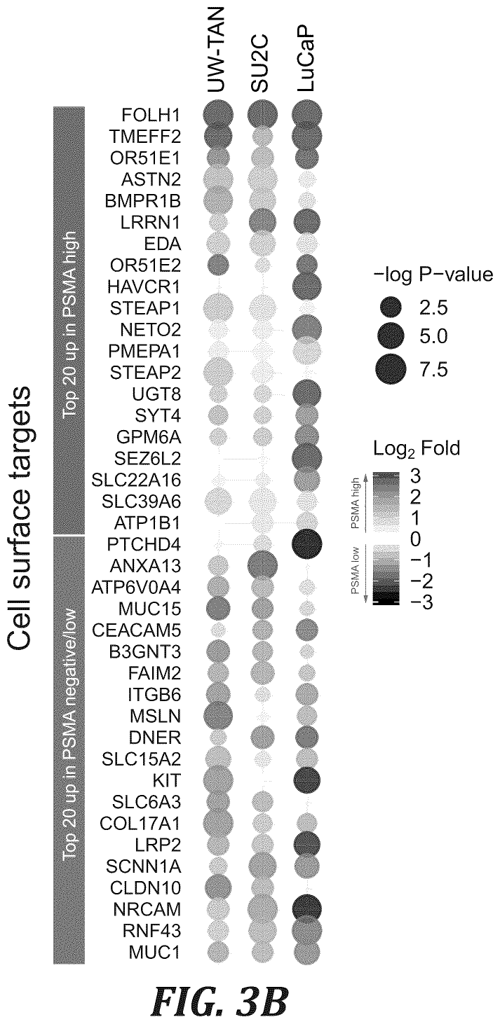

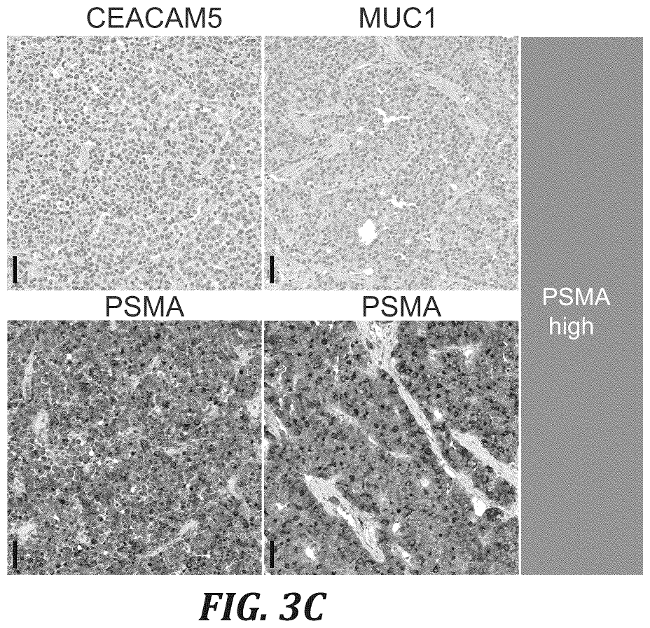

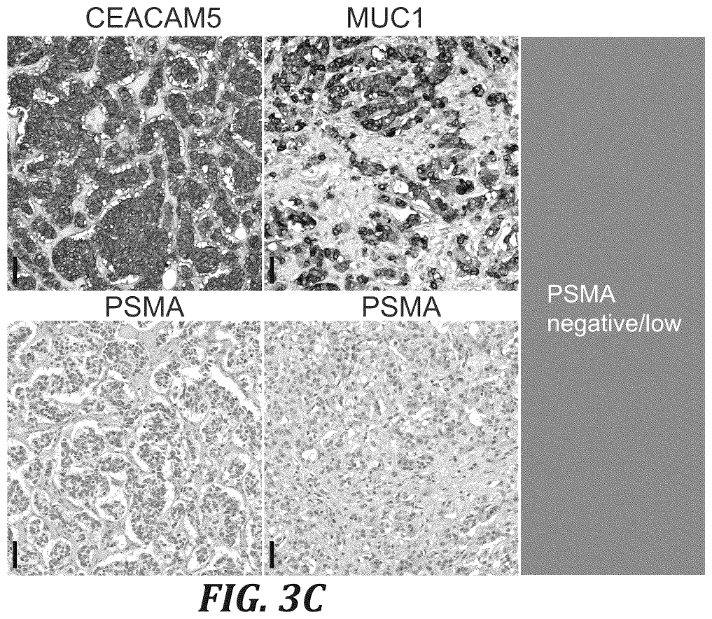

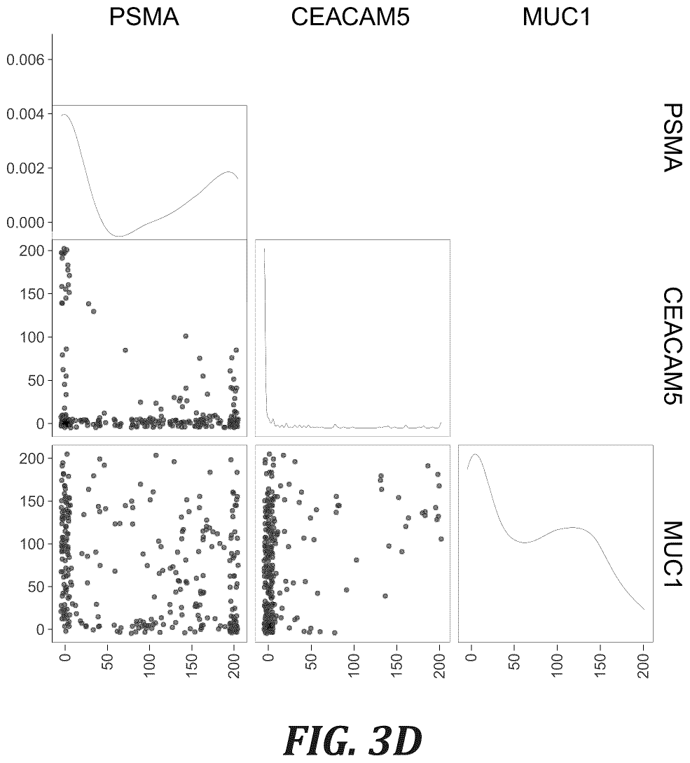

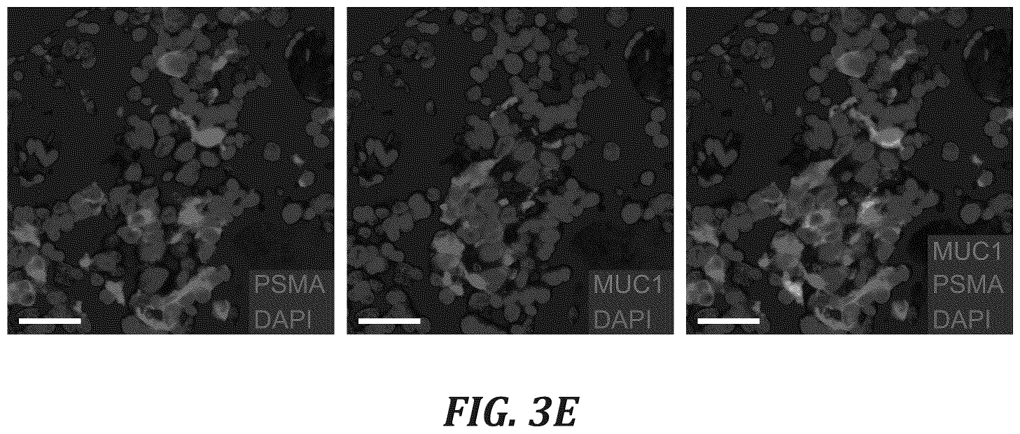

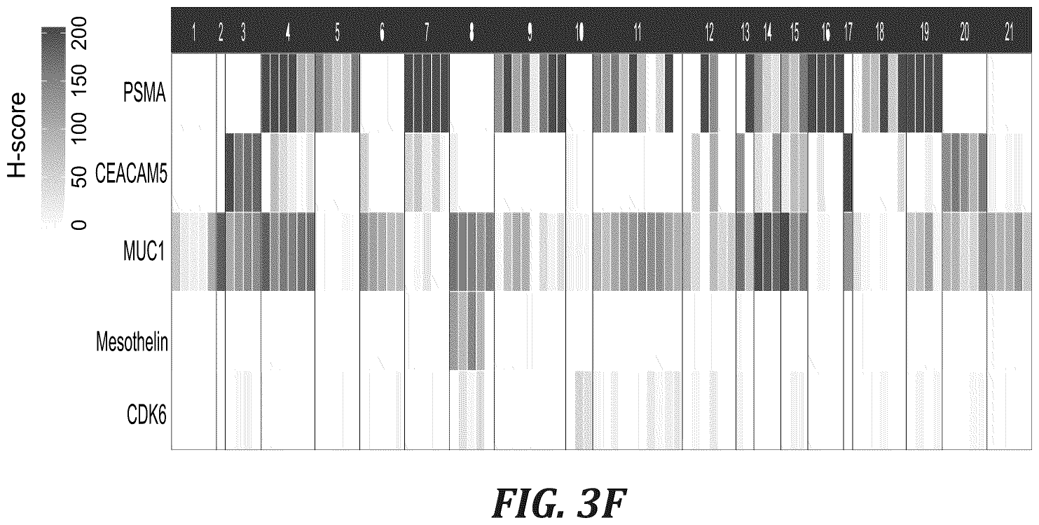

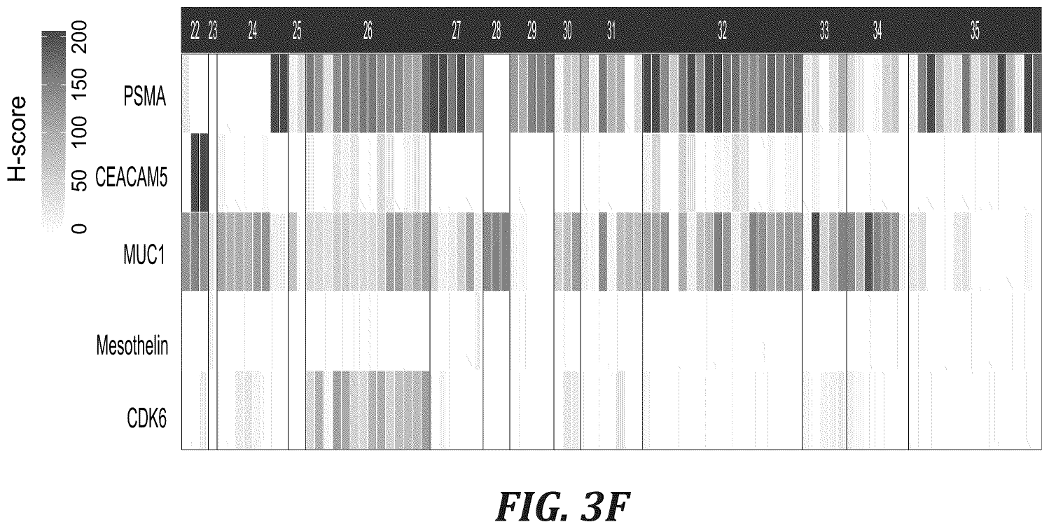

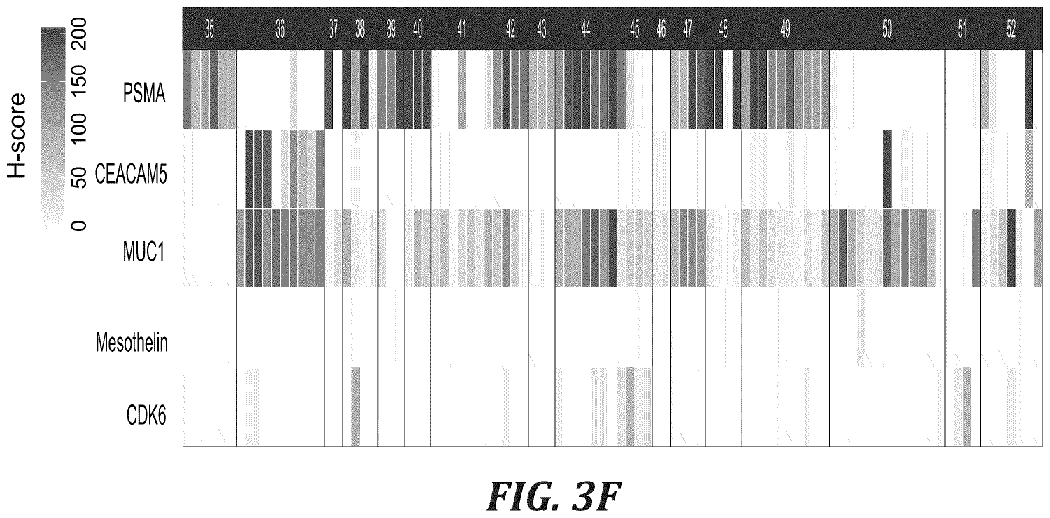

Targetable alterations in PSMA-low/negative tumors. Next, the goal was to investigate whether PSMA low/negative tumors showed shared targetable alterations. To this end, the druggable genome database, a compendium of putative and validated drug targets was used. The top 20 differentially expressed drug targets were determined, which revealed dehydrogenase/reductase family member 9 (DHRS9), Janus kinase 3 (JAK3), megakaryocyte-associated tyrosine kinase (MATK), prostaglandin E synthase (PTGES), alcohol dehydrogenase 1C (ADHIC), mitogen-activated protein kinase 15 (MAPK15), and cyclin-dependent kinase 6 (CDK6) as consistently upregulated in PSMA-low/negative tumors across all cohorts (FIG. 3A and Table 1). Given the role of PSMA as a target for antibody-based and cellular therapies, the expression differences of 409 cell surface proteins previously described in the Cancer Surfaceome Atlas were further assessed. Consistently increased expression of several validated cell surface targets was observed on the mRNA level, including mucin 1 (MUC1, also known as epithelial membrane antigen [EMA]), mesothelin (MSLN), and carcinoembryonic antigen-related cell adhesion molecule 5 (CEACAM5) in PSMA-low/negative tumors (FIG. 3B).

TABLE 1
Differentially expressed cell surface targets in UW-TAN, SU2C and LuCaP samples. Positive
logFC indicates higher in PSMA-high; negative logFC indicates higher in PSMA-low.

| [symbol] ataset | | [symbol] | [symbol] | [symbol] | | | [symbol] | [symbol] | [symbol] | | | [symbol] | [symbol] | [symbol] |
|---|---|---|---|---|---|---|---|---|---|---|---|---|---|---|
| SYMBO [symbol] | MAP | [symbol] | [symbol] | [symbol] | SYMBO [symbol] | MAP | [symbol] | [symbol] | [symbol] | SYMBO [symbol] | MAP | [symbol] | [symbol] | [symbol] |
| OLH1 | 1p11.12 | .49 | .72 | .77 | TCN1 | p13.1- | .47 | 0.54 | .76 | UC20 | q29 | .01 | 0.88 | 0.24 |
| | | | | | | p12 | | | | | | | | |
| STN2 | q33.1 | .56 | .28 | .67 | MODL1 | 1q22.3 | .16 | 0.37 | .69 | NFRSF12A | 6p13.3 | 0.39 | 0.48 | .15 |
| TEAP1 | q21.13 | .31 | .85 | .44 | LT1 | 3q12.3 | .28 | 0.19 | | TPRN2 | q36.3 | 0.14 | .09 | 1.92 |
| RRN1 | p26.2 | .21 | .69 | .92 | LT3 | 3q12.2 | 0.29 | .60 | | LDN3 | q11.23 | 0.25 | 0.57 | 0.17 |
| MEFF2 | q32.3 | .06 | .70 | .76 | PLP1 | 9q13.12 | 0.21 | 0.15 | .82 | LL3 | 9q13.2 | 0.43 | .07 | 0.82 |
| MPR1B | q22.3 | .91 | .34 | .82 | D83 | p23 | .13 | .38 | 0.73 | HRNA5 | 5q25.1 | 0.02 | 0.70 | 0.14 |
| ETO2 | 6q12.1 | .56 | .60 | .75 | LA-DOA | p21.32 | 0.31 | .35 | | PN | 6p11.2 | 0.64 | 0.01 | |
| MEPA1 | 0q13.31 | .60 | .44 | .08 | SPG5 | p21.31 | .14 | .55 | 0.59 | LPPR1 | q31.1 | 0.74 | 0.05 | 1.33 |
| TEAP2 | q21.13 | .32 | .61 | .32 | ECTIN1 | 1q23.3 | 0.05 | 0.17 | .34 | D24 | q21 | .16 | 0.56 | 1.15 |
| ODXL2 | q21.3 | .52 | .21 | .84 | LA-DRA | p21.32 | 0.35 | .60 | 0.56 | PR35 | q37.3 | 0.33 | 0.48 | 0.05 |
| GT8 | q26 | .20 | .04 | .94 | DNRB | 3q22.3 | .43 | 0.37 | | | | | 0.41 | |
| ARCKS | q21 | .35 | .32 | .90 | LPP2 | 9p13.3 | 0.17 | .21 | .03 | AC3 | 7q25.3 | 0.28 | 0.22 | 0.50 |
| PM6A | q34.2 | .22 | .29 | .55 | RXN2 | 1q13.1 | 0.39 | 0.21 | .43 | LEK2 | 4q23.3- | 0.32 | 0.24 | 0.34 |
| | | | | | | | | | | | q24.1 | | | |
| EZ6L2 | 6p11.2 | 0.05 | .30 | .03 | LC29A4 | p22.1 | 0.02 | 0.27 | .46 | LC1 | 2q13.33 | 0.32 | 0.76 | |
| LC22A16 | q21; | .43 | .60 | .26 | UP210 | p25.1 | 0.18 | .09 | .09 | LDN2 | q22.3 | 0.47 | 1.25 | .16 |
| | 6q21-q22.1 | | | | | | | | | | | | | |
| LC39A6 | 8q12.2 | .05 | .72 | 0.52 | PR143 | p22.2 | .22 | .31 | 0.56 | HY1 | 1q23.3 | 0.51 | 0.11 | |
| OLM1 | q21.33 | .94 | .38 | 0.25 | PK2 | 1q23.3 | 0.24 | 0.24 | .56 | MCN1 | q25.3- | 0.27 | 0.43 | |
| | | | | | | | | | | | q31.1 | | | |
| LC10A4 | p11 | .09 | 0.10 | .97 | LRG2 | q34 | 0.43 | .34 | .07 | Y75 | q24.2 | 0.54 | .24 | 1.26 |
| RIK4 | 1q23.3 | .03 | .62 | .32 | CT | 3q32.1 | .57 | 0.01 | 0.78 | AP43 | q13.31 | 0.75 | 0.14 | |
| ROCR | 0q11.22 | .02 | .20 | .27 | D3D | 1q23.3 | 0.76 | .68 | | D33 | 9q13.41 | 0.47 | 0.15 | |
| DGRL3 | q13.1 | 0.43 | .43 | .38 | D38 | p15.32 | 0.54 | .61 | | DAM2 | p11.22 | 0.55 | 0.47 | 1.41 |
| IK3AP1 | 0q24.1 | 0.22 | .15 | .90 | D19 | 6p11.2 | 0.62 | .46 | .03 | GRN | p36.33 | 0.15 | 0.41 | 0.28 |
| USP15 | 0q11.21 | 0.07 | 0.09 | .79 | AG3 | 2p13.31 | 0.72 | .12 | .96 | RIA3 | q25 | 1.06 | 0.22 | |
| ET | q31.2 | 0.12 | 0.37 | .10 | LC16A4 | p13.3 | .05 | 0.19 | .11 | XADR | 1q21.1 | 0.05 | 0.32 | 0.58 |
| CK | p35.2 | 0.72 | .06 | .41 | LC44A3 | p21.3 | .12 | 0.03 | 0.20 | SG | 9p13.3 | 0.32 | 0.03 | 0.18 |
| MEM37 | q14.2 | 0.47 | 0.52 | .43 | QP4 | 8q11.2 | 0.50 | .22 | .10 | JB2 | 3q12.11 | 0.04 | 0.97 | 0.57 |
| RROS | q29 | 0.47 | 0.25 | .05 | NFRSF13B | 7p11.2 | 0.53 | .35 | | YPA | q31.21 | 0.36 | 1.23 | |
| HRNA7 | 5q13.3 | 0.06 | 0.81 | .93 | RM8 | q31.33 | 0.27 | .19 | | INGO1 | 5q24.3 | 0.68 | 0.88 | .35 |
| PRC5B | 6p12.3 | 0.16 | 0.37 | .69 | PCAM | p21 | 0.94 | .23 | 0.26 | TP6V0A4 | q34 | 1.26 | 1.09 | .87 |
| RIN2D | 9q13.33 | 0.55 | 0.42 | .84 | TR3A | 1q23.2 | 0.48 | .28 | | ISS1R | 9p13.3 | 1.17 | 0.36 | 0.38 |
| ACNG4 | 7q24.2 | .16 | 1.69 | .89 | RSS21 | 6p13.3 | .05 | 0.21 | | RC | q24.3 | 0.51 | 1.06 | .26 |
| MEM106A | 7q21.31 | 0.84 | .12 | .39 | ISP2 | 5q15.1 | .13 | 0.15 | 0.17 | PR37L1 | q32.1 | 0.24 | 0.63 | 0.43 |
| TP1B2 | 7p13.1 | 0.25 | 0.73 | .67 | ILRB4 | 9q13.42 | 0.36 | .19 | | ABRA3 | q28 | 1.93 | 0.34 | 0.32 |
| HBG | q22 | 0.86 | 0.68 | .44 | SMD3 | q23.3 | 0.24 | .49 | 0.69 | SD17B2 | 6q23.3 | 0.26 | 1.35 | 0.69 |
| ERPINA5 | 4q32.13 | 0.77 | 1.64 | .66 | LRT1 | 1q13.1 | 0.05 | 0.10 | 0.04 | AB25 | q22 | 0.20 | 0.14 | 0.96 |
| DA | q13.1 | .24 | .19 | .81 | MPRSS2 | 1q22.3 | .13 | .17 | 0.38 | DH6 | p13.3 | 0.17 | 0.54 | |
| R51E1 | 1p15.4 | .39 | .68 | .90 | PM6B | p22.2 | .42 | 0.42 | 0.17 | LC5A10 | 7p11.2 | 0.52 | 0.27 | |
| YT4 | 8q12.3 | .53 | .22 | .23 | AS1 | 9q13.41 | 0.31 | .11 | | HAG | p12.3 | 0.38 | 1.33 | |
| SG2 | 8q12.1 | .38 | .75 | .25 | NFRSF17 | 6p13.13 | 0.51 | .29 | | UC15 | 1p14.2 | 1.91 | 1.48 | .02 |
| R51E2 | 1p15.4 | .70 | .94 | .85 | CKAP1L | 2q13.13- | 0.37 | .26 | | M4SF5 | 7p13.2 | 1.06 | | |
| | | | | | | q13.2 | | | | | | | | |
| TP1B1 | q24.2 | .10 | .70 | .10 | ELSR1 | 2q13.31 | .20 | 0.30 | 0.13 | EACAM6 | 9q13.2 | 0.20 | 1.40 | 0.71 |
| LC44A4 | p21.33 | .88 | .40 | .10 | LAMF9 | q23.2 | 0.24 | .02 | | PINT1 | 5q15.1 | 0.05 | 0.26 | 0.61 |
| ANT1 | 7q25.3 | .37 | .15 | .48 | LRT3 | 0p12.1 | 0.32 | 0.54 | .83 | GT1 | 2q11.23 | 0.25 | 0.63 | 0.84 |
| CPP | q22.1 | .06 | .77 | .06 | PR160 | q26.2 | .43 | .08 | 0.68 | EC | p12-p11 | 0.39 | 0.18 | 0.10 |
| PR137B | q42.3 | .18 | .11 | .09 | D63 | 2q13.2 | 0.03 | .11 | 0.35 | DCP1 | p21.31 | 0.10 | 0.62 | 0.91 |
| BCC11 | 6q12.1 | .61 | .45 | .10 | HRNA3 | 5q25.1 | .32 | 0.78 | .18 | LA-DMA | p21.32 | 0.74 | .05 | 0.71 |
| LTRN | p22.2 | .86 | .09 | .63 | PRC5A | 2p13.1 | 0.37 | 0.06 | 0.06 | NDC10 | p36.33 | 0.72 | 0.30 | 0.67 |
| OMK | p11.21 | .12 | .07 | .13 | ISP3 | p36.22 | 0.13 | 0.32 | .08 | ERINC2 | p35.2 | 0.31 | 0.40 | 0.46 |
| LC18A1 | p21.3 | .32 | .98 | | LC39A8 | q24 | .31 | 0.16 | 0.48 | JB1 | q13.1 | 0.62 | 0.67 | 1.06 |
| LCO5A1 | q13.3 | 0.06 | .70 | .51 | LAMF8 | q23.2 | 0.84 | .51 | | LC34A2 | p15.2 | 1.76 | 0.25 | |
| ADM4 | 9q13.31 | .16 | .30 | .27 | A9 | p13.3 | 0.52 | 0.65 | .71 | LC1A3 | p13.2 | 0.27 | 0.59 | 1.20 |
| LGN3 | q13.1 | .46 | .34 | .51 | GFR4 | q35.2 | 0.40 | 0.33 | .48 | REMEN2 | 6p13.3 | 1.20 | 0.81 | .04 |
| LC3A1 | p21 | .43 | .74 | .53 | DPN | p36.21 | 0.28 | 0.05 | | EMA5B | q21.1 | 0.37 | 0.57 | 1.43 |
| GSF3 | p13.1 | .50 | .29 | .07 | LD4 | 4q32.33 | 0.68 | .45 | | YT6 | p13.2 | 1.36 | 0.23 | |
| LGN1 | q26.31 | .41 | .86 | .23 | TGA3 | 7q21.33 | 0.23 | 0.17 | .38 | RRC15 | q29 | 1.56 | 0.81 | |
| DH1 | 6q22.1 | .22 | .16 | .52 | ADM1 | 1q23.3 | 0.23 | 0.02 | 0.27 | TRA6 | 5q24.1 | 1.34 | 0.11 | |
| EU1 | p21.33 | .20 | .02 | .21 | RRN2 | q32.1 | 0.10 | 0.40 | .23 | EACAM5 | 9q13.2 | 0.59 | 1.24 | 1.77 |
| LC17A5 | q13 | .27 | .37 | 0.03 | SPAN32 | 1p15.5 | 0.31 | .02 | | 3GNT3 | 9p13.11 | 1.54 | 1.16 | 0.70 |
| TRK2 | q21.33 | .06 | .34 | .98 | RB2 | q33.3 | 0.32 | .34 | 0.49 | DKRB1 | 4q32.2 | 0.65 | 0.54 | 0.98 |
| DC3 | p35.2 | .10 | .07 | .51 | 1CAM | q28 | 0.68 | 0.18 | .41 | MPDL3B | p35.3 | 0.73 | 0.27 | 0.56 |
| UC12 | q22.1 | .54 | .40 | .54 | NFRSF9 | p36.23 | 0.44 | .09 | | FSD12 | 9p13.3 | 0.29 | 0.37 | 0.43 |
| LC12A8 | q21.2 | .13 | .07 | .44 | LK1 | 4q32.2 | 0.36 | 0.08 | | CAM1 | 1q23.2 | 0.98 | 0.69 | |
| TK7 | p21.1 | 0.32 | .51 | .21 | UC16 | 9p13.2 | 0.37 | 0.28 | .05 | NC5B | 0q22.1 | 0.41 | 0.68 | 0.80 |
| PAR3 | p22.3 | .18 | 0.17 | .97 | NAS | 0q13.32 | 0.17 | 0.06 | .15 | MEM200C | 8p11.31 | 0.78 | 0.73 | |
| UC13 | q21.2 | .19 | .21 | .31 | PR17 | q14.3 | .07 | 0.39 | | CNS1 | 0q13.12 | 1.02 | 0.30 | |
| PLP2 | 1q24.3 | .44 | .07 | 0.01 | MP14 | 4q11.2 | 0.64 | 0.23 | .45 | LC16A11 | 7p13.1 | 1.24 | 0.60 | 0.69 |
| LBP2 | q25.1 | .20 | 0.01 | .56 | REM2 | p21.1 | 0.28 | 0.03 | | AIM2 | 2q13.12 | 1.17 | 1.18 | 0.92 |
| GFA | p13.3 | .49 | .37 | .20 | A12 | 5q22.2 | 0.59 | 0.20 | .26 | TGB6 | q24.2 | 1.46 | 0.55 | 1.34 |
| LA-DQB1 | p21.32 | 0.42 | .49 | .36 | PINT2 | 9q13.2 | .05 | .02 | 0.29 | NER | q36.3 | 0.83 | 1.49 | 1.96 |
| LECL1 | 2p13.31 | 0.16 | .94 | | LFN1 | p22.3 | 0.14 | 0.63 | .88 | RPM4 | 9q13.33 | 0.64 | 0.64 | 0.82 |
| NTN1 | 2q12 | .32 | .59 | | ULF1 | q13.2- | 0.51 | 0.07 | .07 | M4SF4 | q25.1 | 1.18 | 1.76 | |
| | | | | | | q13.3 | | | | | | | | |
| SPAN10 | 7q25.3 | .32 | 0.07 | .55 | D84 | q23.3 | 0.24 | 0.05 | | LC15A2 | q13.33 | 1.05 | 0.46 | 1.03 |
| DR1 | p21.33 | .27 | .22 | .08 | MP24 | 0q11.22 | 0.01 | 0.10 | 0.48 | LC6A3 | p15.33 | 1.45 | 1.08 | |
| Y6K | q24.3 | .65 | .39 | .05 | CDHGC4 | q31.3 | .24 | 0.16 | 0.86 | LCO1B1 | 2p12.1 | 1.16 | 1.65 | |
| DH10 | p14.2- | .09 | .53 | .42 | ZTS1 | p21.3 | 0.48 | 0.26 | .02 | CNJ16 | 7q24.3 | 1.42 | 0.86 | |
| | p14.1 | | | | | | | | | | | | | |
| DCYAP1R1 | p14.3 | .07 | .62 | | D74 | q33.1 | 0.83 | .21 | .19 | LDN10 | 3q32.1 | 1.65 | 1.07 | |
| AL2 | q24.12 | .51 | .26 | 0.18 | DCY2 | p15.31 | 0.18 | 0.23 | 0.91 | CND2 | q31.31 | .82 | .80 | 2.21 |
| APT | 7q21.31 | .24 | .19 | .39 | LA-DPB1 | p21.32 | 0.60 | .31 | 0.54 | SAMP | q13.31 | 0.01 | .30 | 2.48 |
| TGA4 | q31.3 | 0.09 | .22 | .18 | SR | 9q13.12 | 0.38 | 0.09 | .26 | LDR1 | q13.33 | 0.11 | .41 | 0.65 |
| MEM132A | 1q12.2 | .10 | 0.13 | .92 | PHA8 | p36.12 | 0.90 | 0.24 | .47 | LDN8 | 1q22.11 | .52 | .19 | 2.79 |
| ABRG3 | 5q12 | .39 | .56 | 0.46 | D79B | 7q23.3 | 0.49 | .20 | | RPM8 | q37.1 | .05 | .95 | 3.26 |
| RRN4CL | 1q12.3 | 0.08 | .53 | | L21R | 6p12.1 | 0.70 | .25 | | SPAN1 | p34.1 | .11 | .35 | 1.81 |
| CNK9 | q24.3 | 0.13 | | .70 | MPRSS6 | 2q12.3 | 0.54 | 1.00 | .72 | LC2A12 | q23.2 | .38 | 0.25 | 1.34 |
| D69 | 2p13.31 | 0.46 | .04 | | LC6A17 | p13.3 | 0.71 | 0.75 | .88 | RIG1 | p14.1 | .39 | .18 | 1.12 |
| LC12A2 | q23.3 | .46 | .16 | 0.09 | LA-DQA2 | p21.32 | 0.48 | 0.18 | | A14 | q21.2 | 0.35 | .19 | 1.22 |
| DA | 0q13.12 | 0.12 | .05 | .79 | ZD2 | 7q21.31 | 0.56 | 0.42 | .86 | DC1 | p24.1 | 0.61 | 0.98 | .59 |
| CDHGC3 | q31.3 | .21 | 0.19 | .83 | D80 | q13.33 | 0.50 | .07 | | D44 | 1p13 | 1.77 | .33 | 0.47 |
| BCC4 | 3q32.1 | .03 | .87 | 1.66 | ACE2 | 1q22.2- | .03 | 0.34 | 0.33 | TGER3 | p31.1 | .33 | 1.41 | 0.74 |
| | | | | | | q22.3 | | | | | | | | |
| CNJ10 | q23.2 | 0.07 | .16 | .42 | LC2A1 | p34.2 | 0.01 | 0.02 | 0.36 | OX1 | q22.1 | .04 | .52 | 2.05 |
| MB | q11.1 | .18 | .54 | 0.55 | SHR | 4q31.1 | 0.14 | 0.31 | | T14 | 1q24.3 | .09 | 0.50 | 0.72 |
| LA-DQA1 | p21.32 | 0.25 | .51 | | LC45A2 | p13.2 | 0.89 | .05 | 0.31 | HRNA2 | p21.2 | 0.38 | 0.78 | 1.55 |
| EGF11 | 5q22.31 | .26 | .66 | 0.60 | LCNKB | p36.13 | 1.21 | 0.25 | .80 | LC7A8 | 4q11.2 | 0.08 | 0.46 | 0.98 |
| LDN7 | 7p13.1 | .06 | 0.12 | .35 | TSG | 4q12 | 0.71 | 0.03 | | NC5A | q35.2 | 0.19 | 0.79 | 2.39 |
| LP1 | q22.2 | .30 | .15 | | MMR | q34 | 0.32 | 0.09 | 0.20 | LC39A4 | q24.3 | 0.72 | 0.80 | .29 |
| DH3 | 6q22.1 | 0.01 | .38 | .19 | D52 | p36.11 | 0.83 | .27 | | YPD1 | q21.2 | 0.77 | 1.18 | .41 |
| SPAN12 | q31.31 | .23 | .27 | 0.20 | TYH1 | 9q13.42 | 0.19 | 0.18 | 0.34 | PHB2 | p36.12 | 1.18 | 0.22 | 0.71 |
| TP1A3 | 9q13.2 | 0.66 | .39 | .15 | TPRZ1 | q31.32 | 0.23 | 0.80 | 0.32 | LC3A2 | 1q12.3 | 0.21 | 0.47 | 0.27 |
| MEM30B | 4q23.1 | .31 | .35 | 0.62 | TGAL | 6p11.2 | 0.73 | .25 | | SLN | 6p13.3 | 1.85 | 0.24 | 1.06 |
| ELTF | q29 | 0.12 | 0.17 | .85 | CDH15 | 0q21.1 | 0.86 | 0.04 | | LDN4 | q11.23 | 0.34 | 0.33 | 0.80 |
| RID1 | 0q23.1- | .04 | 0.11 | .88 | ACSTD2 | p32.1 | 0.06 | .10 | 0.64 | CDHB9 | q31.3 | 0.27 | 0.51 | 2.33 |
| | q23.2 | | | | | | | | | | | | | |
| RIA4 | 1q22.3 | .55 | 0.10 | | IT1 | p13.3 | 1.01 | .44 | | CSTAMP | q22.3 | 1.41 | 0.56 | |
| UBN | 0p13 | .21 | 0.42 | .89 | LC7A5 | 6q24.2 | 0.19 | 0.86 | .79 | UC1 | q22 | 1.16 | 1.06 | 1.59 |
| 2RY8 | p22.33; | .13 | .04 | | AIR1 | 9q13.42 | 0.43 | .03 | | ET | 0q11.21 | 0.83 | 0.61 | 3.14 |
| | Yp11.2 | | | | | | | | | | | | | |
| D79A | 9q13.2 | 0.50 | .92 | | MP16 | q21.3 | 0.47 | 0.11 | | OL17A1 | 0q25.1 | 1.47 | 0.85 | 1.05 |
| PC3 | q26.2 | 0.06 | .17 | .24 | LEC12A | 2p13.31 | 0.35 | 0.35 | | M4SF1 | q25.1 | 0.54 | 0.56 | 1.61 |
| TA | p21.33 | 0.38 | .62 | | PNMB | p15.3 | 0.22 | 0.51 | .05 | RP2 | q31.1 | 1.14 | 0.93 | 2.46 |
| IK3CG | q22.3 | 0.02 | .18 | | L13RA2 | q23 | .12 | 0.67 | 0.78 | YO10 | p15.1 | .15 | 0.47 | 1.11 |
| SPAN8 | 2q21.1 | .46 | .04 | 1.28 | Y6E | q24.3 | 0.40 | 0.31 | .04 | TP8B4 | 5q21.2 | 0.29 | 0.57 | 3.18 |
| CNQ2 | 0q13.33 | 1.44 | .72 | .89 | RRC55 | 1q12.1 | 0.17 | 0.46 | | XGR1 | 3q32.1 | 0.52 | 0.58 | 4.56 |
| RB3 | 9p13.3 | .11 | .03 | 0.01 | TLN1 | q23.3 | 0.17 | 0.62 | | TCHD4 | p12.3 | 0.19 | 0.61 | 4.97 |
| D276 | 5q24.1 | .11 | .00 | .04 | HRM1 | 1q12.3 | 0.22 | 0.33 | 0.55 | NXA13 | q24.13 | 0.90 | 1.97 | |
| DC4 | 0q13.12 | 0.11 | .35 | 0.04 | LA-DRB1 | p21.32 | 0.70 | .30 | 1.02 | IT | q12 | 1.46 | .18 | 2.57 |
| | | | | | IGLEC15 | 8q12.3 | 0.25 | 0.53 | | CNN1A | 2p13.31 | 0.84 | 1.46 | 1.55 |
| | | | | | CAM | 9q13.32 | 0.62 | 0.07 | 0.10 | RCAM | q31.1 | 0.86 | 1.31 | 2.67 |
| | | | | | TPRN | q35 | 0.83 | 0.41 | .02 | NF43 | 7q22 | 0.77 | 1.06 | 1.75 |

[symbol]

indicates data missing or illegible when filed

To corroborate these in silico findings, IHC studies were performed for CEACAM5, MUC1, MSLN, and CDK6 in an additional cohort of 52 rapid autopsy cases (FIGS. 3C-3F). While the protein expression of MSLN and CDK6 was restricted to a small number of cases, MUC1 and CEACAM5 were expressed in a large fraction of metastases (FIGS. 3D-3F). Importantly, out of 102 tumors with low/no PSMA expression (H-score <20), 91 (89.2%) showed MUC1 expression (mean H-score=92). Conversely, in the 88 tumors with low/no MUC1 expression, PSMA was expressed in 73 samples (83%) (FIG. 3F). These findings suggest inverse expressions of PSMA and MUC1. In support of this notion, dual immunofluorescence labeling studies in PSMA- and MUC1-positive cases demonstrated that MUC1 and PSMA were expressed in separate cell populations with limited co-expression at the single-cell level (FIG. 3E). Collectively, these findings indicate that tumors with low/negative PSMA expression exhibit distinct transcriptional changes. Furthermore, these results show that MUC1 represents a relevant surface target in PSMA-low/negative tumors.

### Example 11

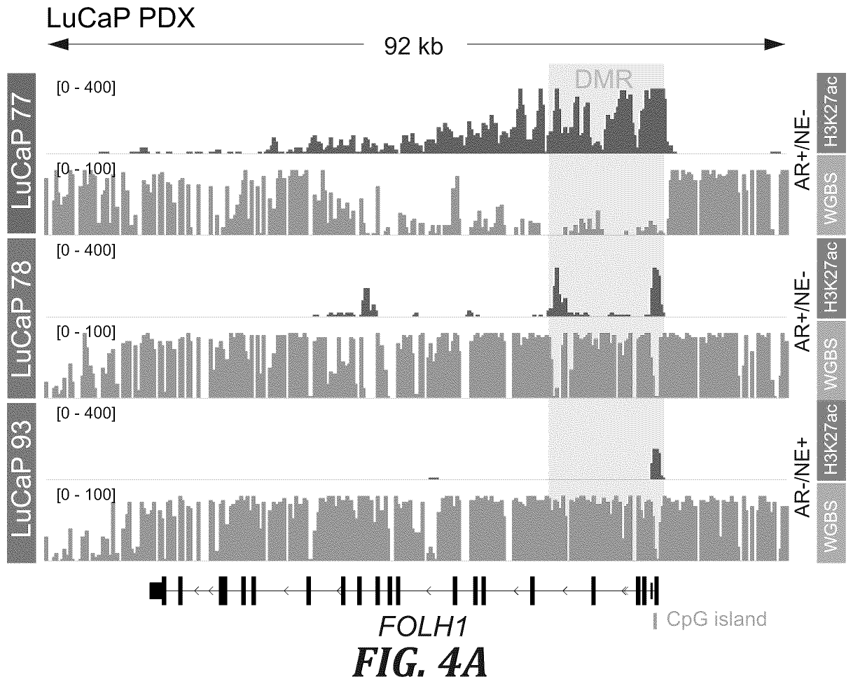

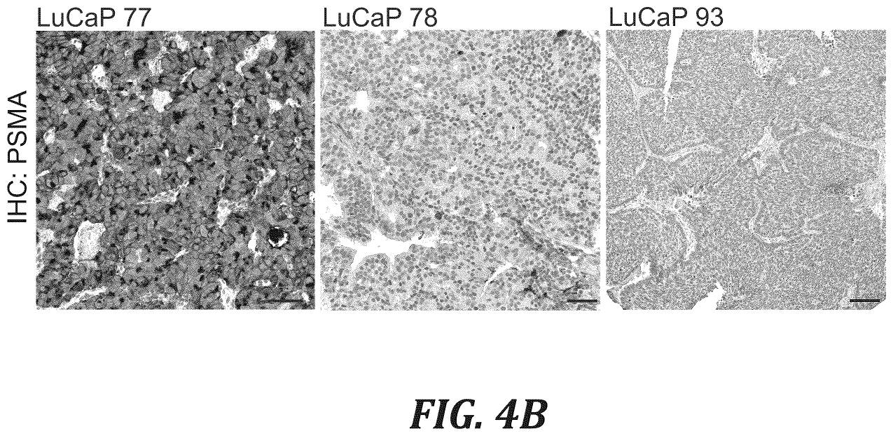

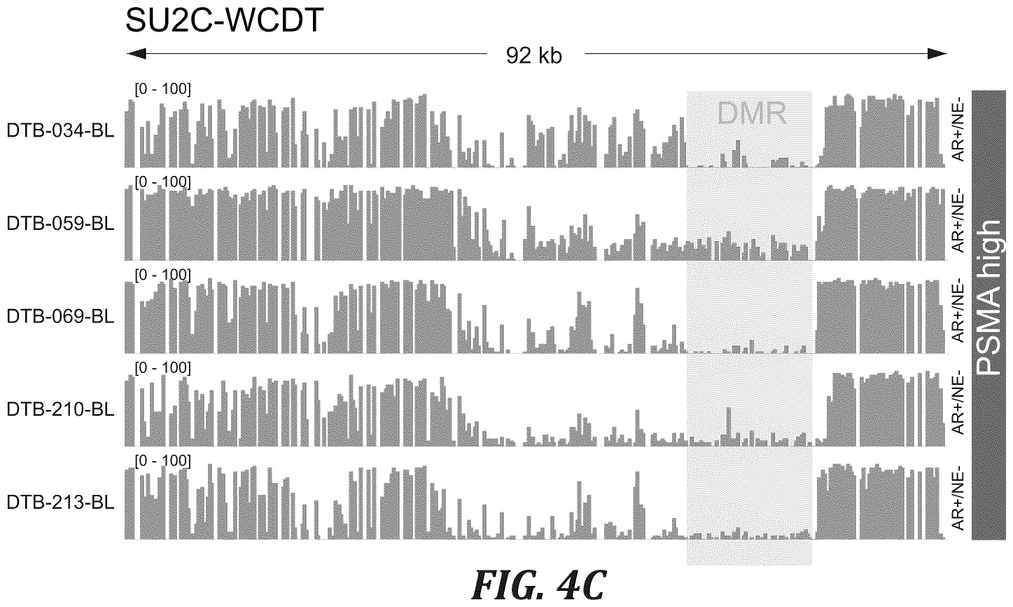

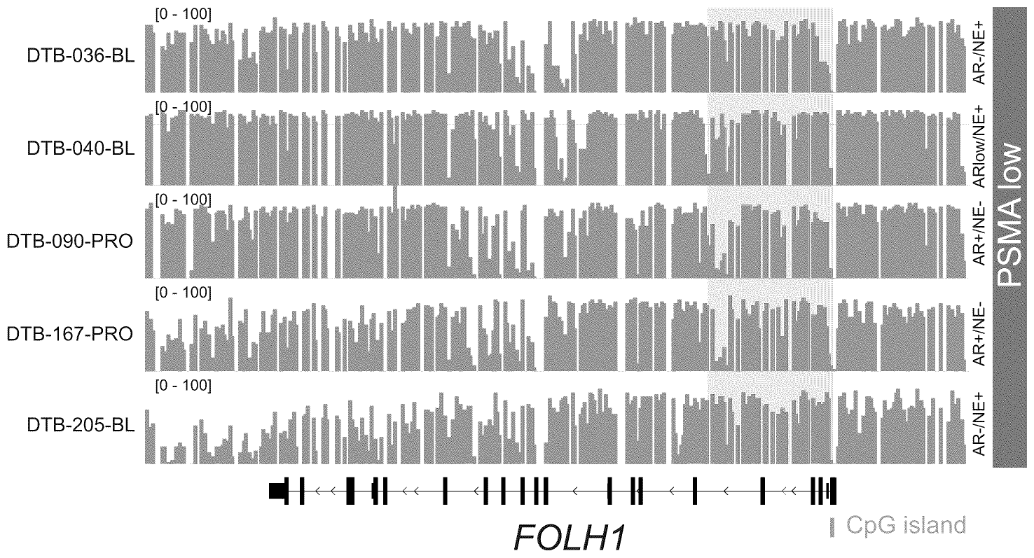

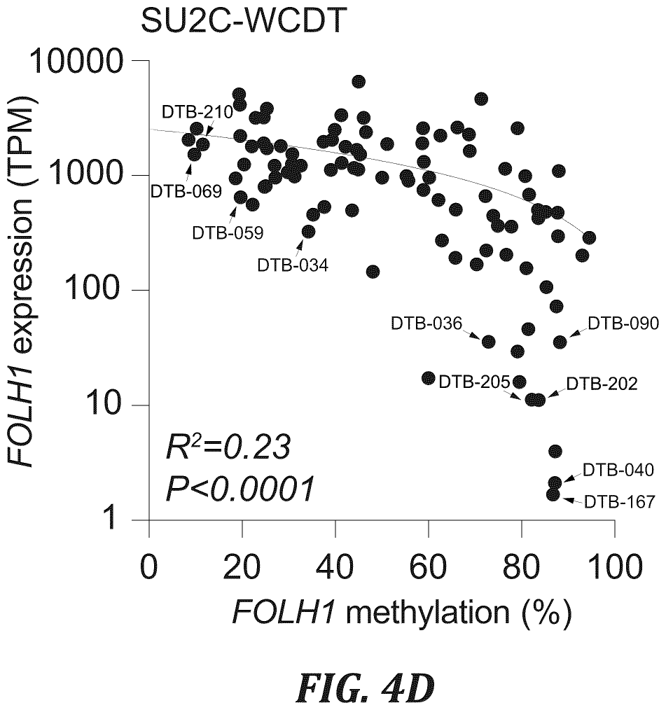

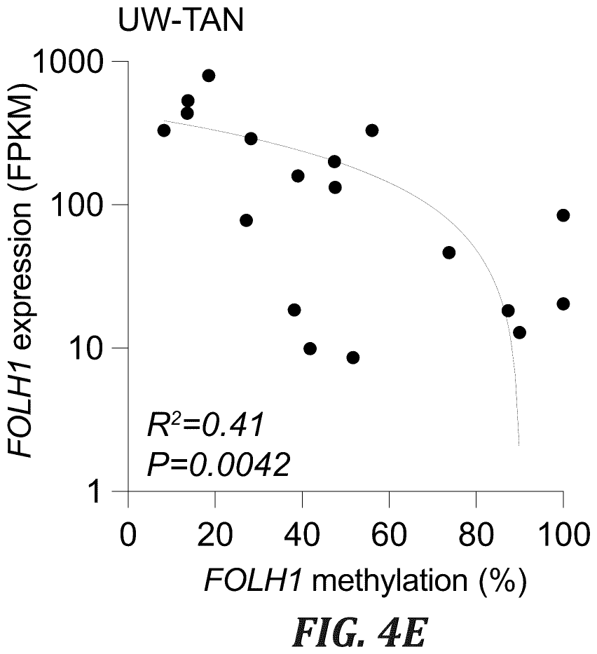

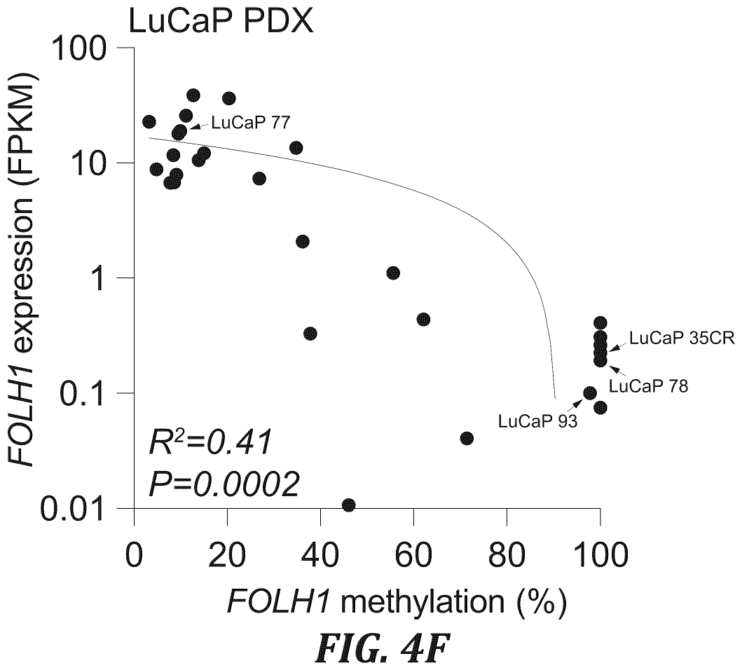

Cooperating epigenetic changes are associated with PSMA silencing. The variability in PSMA expression prompted inquiries to further determine independent of AR mechanisms of PSMA regulation. To this end, first, epigenetic features of the FOLH1 gene locus in LuCaP PDX lines with distinct PSMA protein expression were assessed (FIGS. 4A-4B). Whole-genome bisulfite sequencing (WGBS) identified an approximately 14 kb region of the FOLH1 gene encompassing the first 5 introns, but not a CpG island at the transcriptional start site, which showed CpG hypermethylation in the PSMA-negative PDX lines LuCaP 78 and 93 (FIG. 4A). In PSMA expressing LuCaP 77, this differentially methylated region (DMR) was hypomethylated. Furthermore, leveraging ChIP-seq and ATACSeq chromatin profiles of LuCaP PDX lines, DNA methylation was observed to be inversely correlated with chromatin accessibility and enrichment for histone 3 lysine 27 acetylation (H3K27ac) (FIG. 4A).

To investigate the association between DNA methylation and FOLH1 expression in clinical specimens, a previously published series of CRPC WGBS samples (SU2C-WCDT, n=98) (FIG. 4C) were analyzed. Similar to LuCaP PDX models, a robust difference in FOLH1 gene methylation profiles was observed between PSMA-high and PSMA-low tumors (FIGS. 4C-4D). The tight correlation between PSMA/FOLH1 expression and FOLH1 CpG methylation observed in WGBS studies was further corroborated using a targeted methylation enrichment assay (COMPARE-MS) in additional LuCaP PDX (n=29) and UW-TAN rapid autopsy tissue samples (n=18) (FIGS. 4E-4F). Of note, FOLH1 hypermethylation was observed in both AR+ and AR− tumors, and all AR−/NE+ samples showed CpG hypermethylation (FIG. 4A and FIG. 4C). These findings demonstrate that, independently of AR or NE status, DNA methylation changes are tightly linked to PSMA expression and that FOLH1 gene regulation is controlled by an interplay of repressive (DNA hypermethylation) and active (H3K27ac) chromatin modifications.

### Example 12

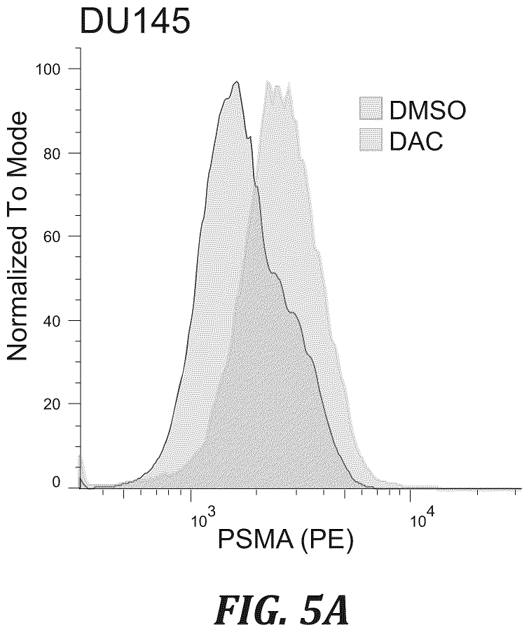

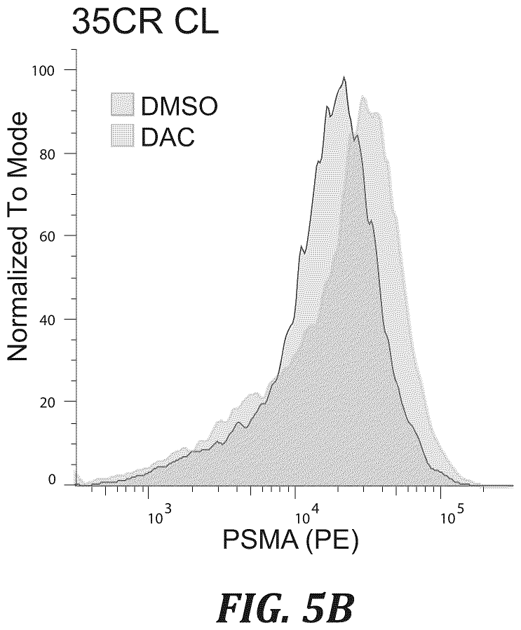

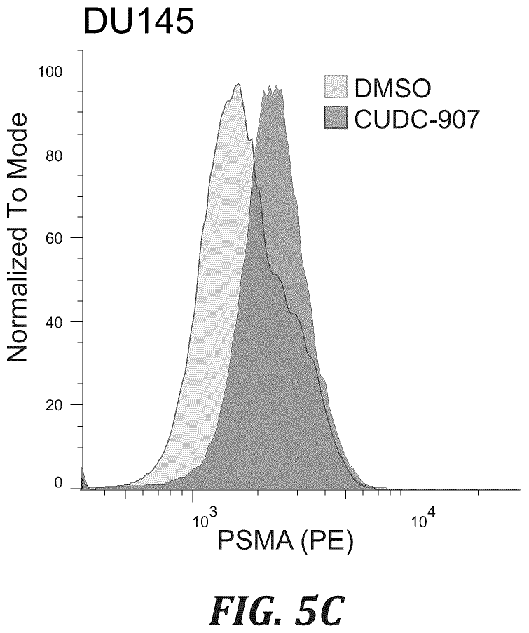

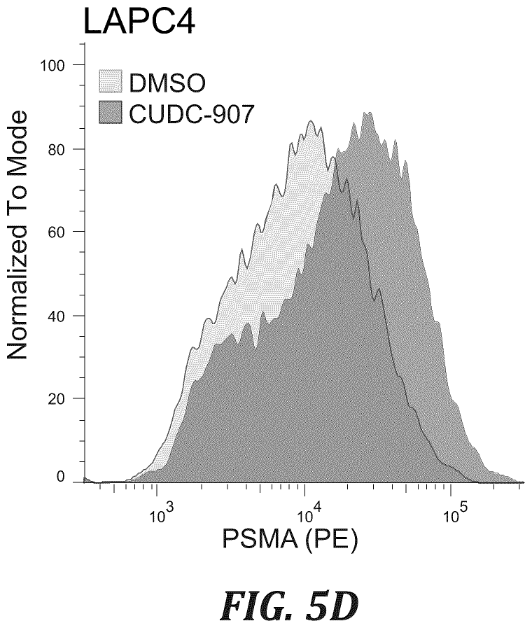

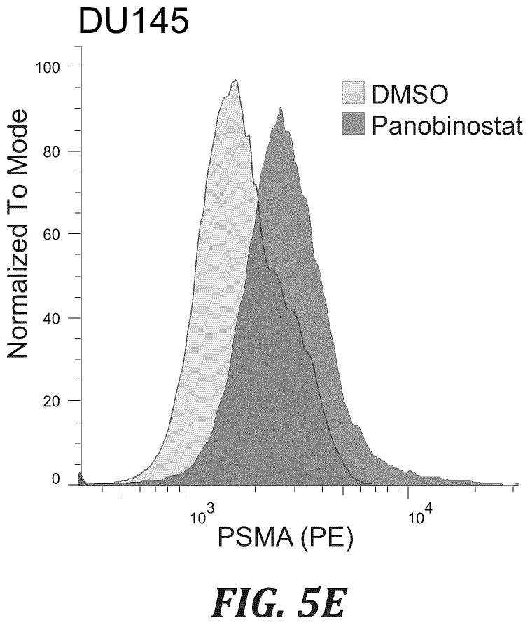

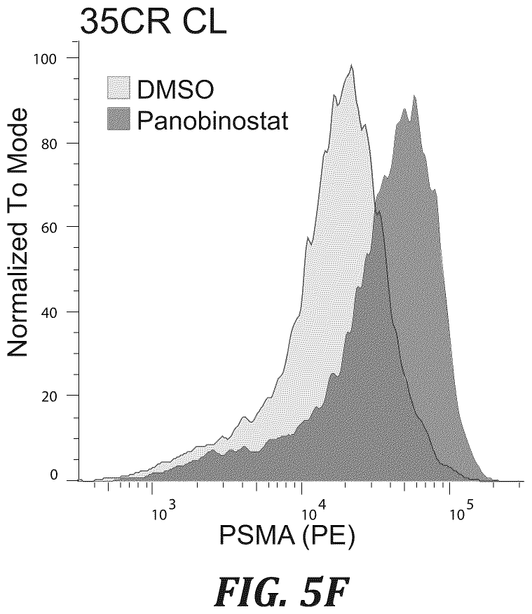

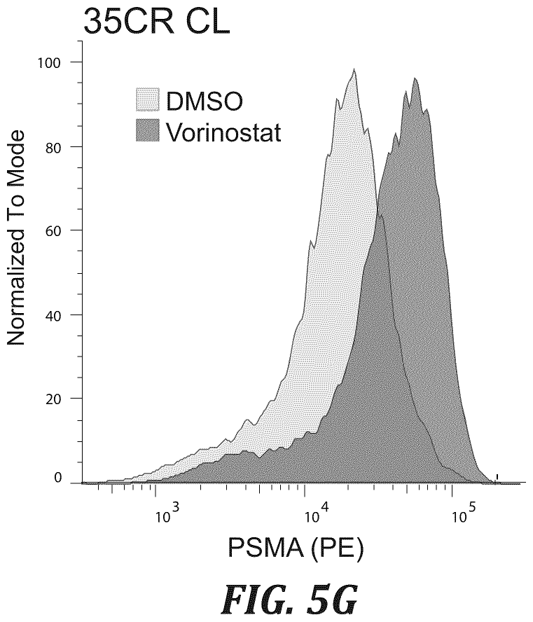

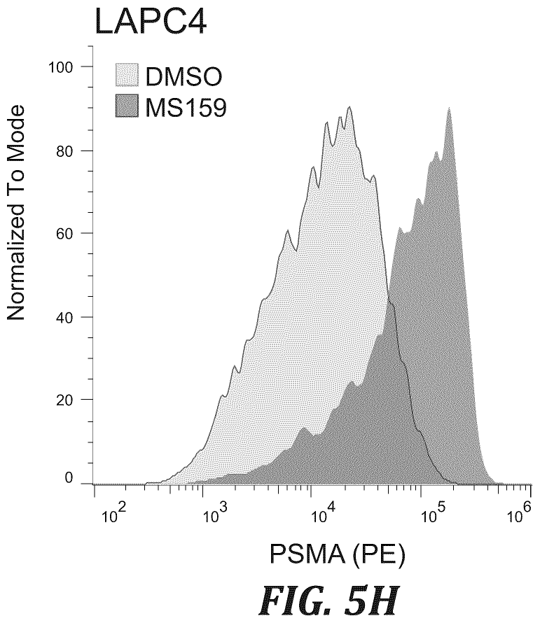

Epigenetic therapies can restore and augment PSMA expression. Epigenetic changes are known to be at least partly reversible. Pharmacologic inhibitors of enzymes involved in DNA methylation (DNA methyltransferases [DNMT]) and histone acetylation (histone deacetylases [HDAC]) have been developed and explored clinically to modulate epigenetic states in cancers. Given these insights into the epigenetic regulation of PSMA that involves the interplay between DNA methylation and histone acetylation, the aim was to test if DNMT inhibitors (DNMTi) or HDAC inhibitors (HDACi) could augment PSMA expression in cell line models (FIGS. 5A-5H). To this end, 3 cell line models (DU145, LAPC4, and LuCaP 35CR CL) were treated with the DNMTi decitabine and pan-HDACi (panobinostat, CUDC-907, vorinostat) and applied 2 orthogonal approaches (flow cytometry and fluorescence immunocytochemistry) and 2 separate monoclonal antibodies (3E6 and LNI-17) to measure PSMA protein expression. Decitabine treatment alone resulted in increased expression in DU145 and LuCaP 35CR CL cells (FIGS. 5A-5B), suggesting that DNMTi is effective in enhancing PSMA expression. HDACi treatment, however, increased PSMA protein expression in all lines, irrespective of baseline PSMA levels (FIGS. 5C-51).

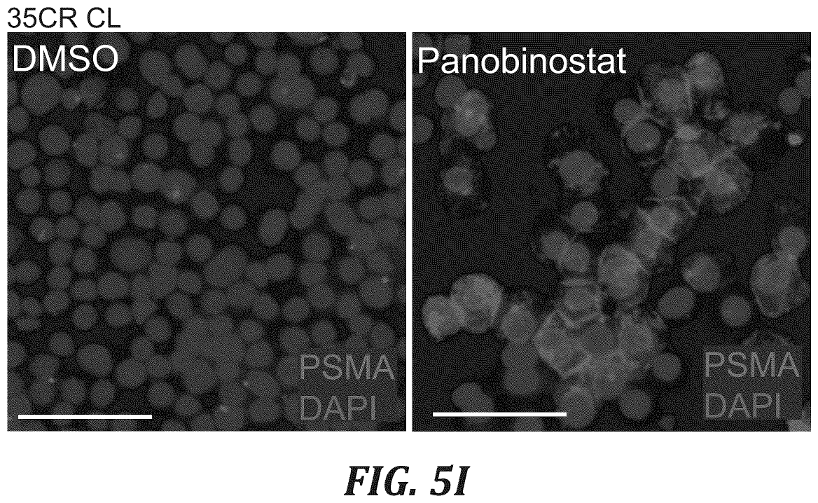

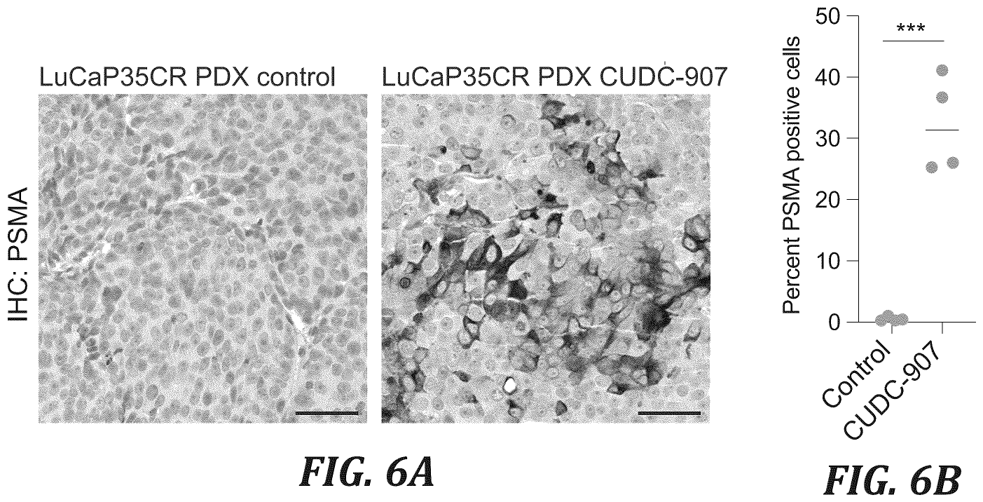

To determine whether these in vitro results would also extend to in vivo responses, mice bearing LuCaP 35CR xenografts (a PDX line in which the FOLH1 locus is methylated, which shows very low/no baseline PSMA expression) were treated with the pan-HDACi CUDC-907. Tumors harvested after 3 weeks of treatment with CUDC-907 showed significantly increased PSMA expression, whereas only very rare positive cells were detected in control-treated animals (FIGS. 6A-6B).

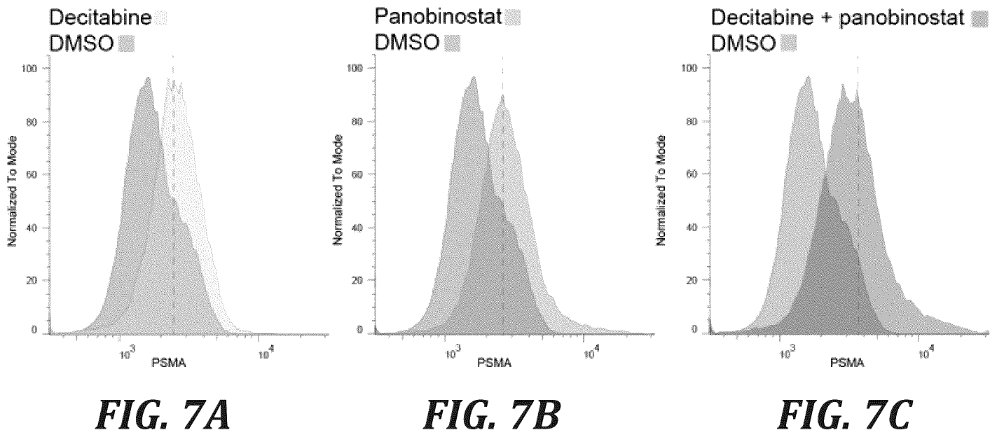

Lastly, to assess whether a DNA methylation inhibitor and histone acetylation inhibitor used together could augment PSMA expression in cell line models, DU145 cell lines were treated with a combination of Decitabine and panobinostat. FIGS. 7A-7C show augmented PSMA expression by combined HDAC and DNMT inhibition. Density plots show PSMA cell surface expression in DU145 with vehicle control (DMSO), decitabine (FIG. 7A), Panobinostat (FIG. 7B), or decitabine and Panobinostat (FIG. 7C). Note the right shift of the modal distribution as well as the longer right sided tail in the combination treatment.

These data suggest that the epigenetic changes repressing PSMA transcription are potentially reversible and that epigenetic drugs can be used to augment PSMA expression.

The embodiments of the invention in which an exclusive property or privilege is claimed are as follows:

1. A method for overcoming resistance to and/or augmenting efficacy of Prostate specific membrane antigen (PSMA)-directed therapies in a subject in need thereof, the method comprising:
administering to the subject an effective amount of at least one first agent capable of modulating the expression of PSMA.

2. The method of claim 1, wherein the subject is human, and wherein the subject has prostate cancer.

3. The method of claim 2, wherein the prostate cancer is a PSMA-negative or PSMA-low prostate cancer, and wherein the method restores and/or upregulates the expression of PSMA.

4. The method of claim 3, wherein the prostate cancer is non-responsive to at least one other therapeutic modality.

5. The method of claim 1, wherein the at least one first agent capable of modulating the expression of PSMA comprises at least one epigenetic modifier, wherein the epigenetic modifier targets and/or modulates the epigenetic state of Folate Hydrolase (FOLH1) gene to upregulate and/or restore the expression of PSMA.

6. The method of claim 5, wherein the epigenetic modifier is selected from a small molecule, a nucleic acid molecule, an expression vector comprising a nucleic acid molecule, and a polypeptide molecule.

7. The method of claim 6, wherein the epigenetic modifier is a small molecule, and wherein the small molecule is selected from a histone deacetylase inhibitor (HDACi), Nuclear SET Domain 2 (NSD2) inhibitor, DNA methyltransferase inhibitor (DNMTi), and combinations thereof.

8. The method of claim 6, wherein the epigenetic modifier is a small molecule, and wherein the small molecule is selected from a combination of (i) histone deacetylase inhibitor (HDACi) and NSD2, (ii) NSD2 and DNA methyltransferase inhibitor (DNMTi), and (iii) HDACi and DNMTi.

9. The method of claim 1, wherein the at least one first agent capable of modifying the expression of PSMA is administered prior to or together with administration of or in conjunction with at least one second agent.

10. The method of claim 9, wherein the at least one second agent comprises a therapeutically effective amount of at least one PSMA-directed therapeutic agent.

11. The method of claim 10, wherein the at least one PSMA-directed therapeutic agent comprises at least one targeting component coupled to at least one therapeutic component.

12. The method of claim 11, wherein the at least one targeting component is selected from a small molecule binder of PSMA, PSMA ligand, PSMA-binding peptide, PSMA-binding aptamer, monoclonal antibodies specific for PSMA, and derivatives thereof.

13. The method of claim 11, wherein the at least one therapeutic component or therapeutic agent is a theranostic agent, wherein the theranostic agent is selected from a radioligand/radionuclide or a cytotoxic agent.

14. The method of claim 11, wherein the at least one PSMA-directed therapy comprises PSMA-targeted radioligand therapy (PSMA-RLT), wherein the radionuclide is selected from alpha-emitters and beta-emitters.

15. A method for treating prostate cancer, the method comprising:
(i) obtaining at least one biological sample from a subject suffering from prostate cancer;
(ii) determining the epigenetic state of FOLH1 gene for the presence of at least one epigenetic modification at the FOLH1 gene locus in the at least one biological sample;
(iii) determining the expression of at least one of Carcinoembryonic antigen-related cell adhesion molecule 5 (CEACAM5), Mucin 1 (MUC1), and Mesothelin (MSLN) in the at least one biological sample; and
(iii) administering to the subject at least one agent.

16. The method of claim 15, wherein the at least one epigenetic modification is selected from differential methylation of FOLH1 locus, gain of CpG methylation, and loss of histone 3 lysine 27 (H3K27) acetylation, and wherein the at least one epigenetic modification is associated with a low or no expression of PSMA.

17. The method of claim 16, wherein the biological sample is determined to overexpress MUC1.

18. The method of claim 17, wherein the at least one agent comprises a therapeutically effective amount of at least one MUC1-targeting therapeutic agent.

19. The method of claim 16, wherein the at least one agent comprises an epigenetic modifier, wherein the epigenetic modifier targets and/or modulates the epigenetic state of FOLH1 gene; and wherein the epigenetic modifier upregulates and/or restores the expression of PSMA.

20. The method of claim 19, wherein the epigenetic modifier is a small molecule, and wherein the small molecule is selected from histone deacetylase inhibitor (HDACi), DNA methyltransferase inhibitor (DNMTi), NSD2, and combinations thereof.

21. The method of claim 19 further comprising administering to the subject a therapeutically effective amount of at least one PSMA-targeted therapeutic agent.

22. The method of claim 21, wherein the at least one PSMA-targeted therapeutic agent comprises at least one targeting component coupled to at least one therapeutic component, wherein the at least one targeting component is selected from small molecule binders of PSMA, PSMA ligand, PSMA-binding peptide, PSMA-binding aptamer, and monoclonal antibodies specific for PSMA, and derivatives thereof, and wherein the at least one therapeutic component is selected from a radioligand/radionuclide or a cytotoxic agent.

23. The method of claim 22, wherein the at least one agent and the at least one PSMA-targeted therapeutic agent are administered sequentially or simultaneously.

24. The method of claim 15, wherein the biological sample is a cell-free sample, and wherein the cell-free sample comprises blood, serum, or plasma.

25. The method of claim 15, wherein the prostate cancer is a metastatic castration resistant prostate cancer.

26. A method for treating prostate cancer comprising:
(a) selecting and sensitizing a subject in need thereof to a PSMA-directed therapeutic agent; and
(b) administering a therapeutically effective amount of at least one PSMA-directed therapeutic agent,
wherein the step of selecting and sensitizing the subject comprises:
(i) obtaining a first biological sample from the subject;
(ii) determining at least one epigenetic modification of the FOLH1 locus in the first biological sample to obtain a first assessment of PSMA expression;
(iii) administering to the subject an effective amount of at least one epigenetic modifier;
(iv) obtaining a second biological sample; and
(v) determining a change in the at least one epigenetic modification of the FOLH1 locus in the second biological sample to obtain a second assessment of PSMA expression,
wherein the first assessment of PSMA expression comprises a low or no expression of PSMA, and the second assessment of PSMA expression comprises a restoration and/or upregulation of PSMA expression.

27. The method of claim 26, wherein the first and the second biological sample is a cell-free sample, and wherein the first and second biological sample is selected from blood, serum, or plasma.

28. The method of claim 26, wherein the at least one epigenetic modification of the FOLH1 locus determined in the first biological sample is selected from differential methylation of FOLH1 locus, gain of CpG methylation, and loss of histone 3 lysine 27 (H3K27) acetylation.

29. The method of claim 26, wherein the at least one epigenetic modifier is selected from a small molecule, a nucleic acid molecule, an expression vector comprising a nucleic acid molecule, and a polypeptide molecule.

30. The method of claim 29, wherein the at least one epigenetic modifier is a small molecule, and wherein the small molecule is selected from a histone deacetylase inhibitor (HDACi), DNA methyltransferase inhibitor (DNMTi), NSD2, and combinations thereof.

31. The method of claim 26, wherein the at least one PSMA-targeted therapeutic agent comprises at least one targeting component coupled to at least one therapeutic component, wherein the at least one targeting component is selected from a small molecule binder of PSMA, a PSMA ligand, a PSMA-binding peptide, a PSMA-binding aptamer, and a monoclonal antibody specific for PSMA, and derivatives thereof, and wherein the at least one therapeutic component comprises a radioligand/radionuclide.

32. The method of claim 31, wherein the radionuclide is selected from alpha-emitters and/or beta-emitters.

33. The method of claim 26, wherein the at least one PSMA-targeted therapeutic agent comprises at least one targeting component coupled to at least one therapeutic component, wherein the at least one targeting component is selected from a small molecule binder of PSMA, a PSMA ligand, a PSMA-binding peptide, a PSMA-binding aptamer, and a monoclonal antibody specific for PSMA, and derivatives thereof, and wherein the at least one therapeutic component comprises a cytotoxic agent.

34. The method of claim 26, wherein the prostate cancer is a metastatic castration resistant prostate cancer.
